(19) United States
(12) Patent Application Publication
(10) Pub. No.: US 2009/0018106 A1
(43) Pub. Date: Jan. 15, 2009

(54) INTRAVENOUS FORMULATIONS OF PYRIDOXAL 5'- PHOSPHATE AND METHOD OF PREPARATION

(75) Inventor: Albert Friesen, Winnipeg (CA)

Correspondence Address: MERCHANT & GOULD PC, P.O. BOX 2903, MINNEAPOLIS, MN, 55402-0903, US

(73) Assignee: Medicure International Inc., St. James, West Indies (BB)

(21) Appl. No.: 11/910,110

(22) PCT Filed: Mar. 30, 2006
(86) PCT No.: PCT/CA2006/000467

§ 371 Date: Apr. 3, 2008

(30) Foreign Application Priority Data
Mar. 30, 2005 (CA) 2,503,087

(51) Int. Cl. A61K 31/675; C07F 9/576; A61P 1/08

(52) U.S. Cl. 514/89; 546/21

(57) ABSTRACT

The present invention provides a lyophilized formulation of pyridoxal 5′-phosphate and a method of making the same. The present invention also provides an injectable formulation of pyridoxal 5′-phosphate reconstituted from the lyophilized formulation. The present invention further provides uses of the lyophilized and injectable formulations.

### FIELD OF INVENTION

The present invention relates to pharmaceutical formulations of pyridoxal 5′phosphate, and in particular formulations suitable for intravenous administration.

### BACKGROUND

Pyridoxal 5′-phosphate (P5P) is useful for the treatment and prevention of a variety of diseases such as hypertension, cerebrovascular disorders, cardiovascular disorders and diabetes. See for example U.S. Pat. Nos. 6,051,587; 6,417,204; 6,548,519; 6,586,414; 6,605,612; 6,667,315; 6,780,997; 6,677,356; 6,489,348; and 6,043,259.

Intravenous formulations of pyridoxal 5′-phosphate are known in the prior art. However, the stability of aqueous solutions of pyridoxal 5′-phosphate is poor. As well, it is highly light sensitive in solution, and to a lesser degree also in powder form. As such, prior art intravenous formulations of pyridoxal 5′-phosphate required the inclusion of preservatives for improving stability and for increasing shelf life. However, such preservatives often give rise to undesirable side effects and have not addressed the light sensitivity of pyridoxal 5′-phosphate. As well, such preservatives are often unsuitable for use in regulatory approval.

The present invention provides novel intravenous formulations of pyridoxal 5′-phosphate which are less susceptible to degradation and which do not require the inclusion of preservatives. The intravenous formulations exhibit improved pharmacokinetic profiles.

### SUMMARY OF INVENTION

In one aspect, the present invention provides a lyophilized formulation of pyridoxal 5′-phosphate having been prepared by lyophilizing a frozen sterile aqueous solution of pyridoxal 5′-phosphate in a concentration higher than a supplement concentration and sodium hydroxide.

In an embodiment of the invention, the pH of the solution is between 7.0 and 7.3.

In another embodiment of the invention, the lyophilized formulation further comprises mannitol.

In a further aspect, the present invention provides an injectable formulation containing pyridoxal 5′-phosphate, reconstituted from a lyophilized formulation according to the invention, using a sterile carrier suitable for intravenous administration.

In an embodiment of the invention, the sterile carrier is water for injection.

In a further aspect, the present invention provides a process for preparing a lyophilized formulation of pyridoxal 5′-phosphate comprising the steps: (a) preparing a sterile solution comprising pyridoxal 5′-phosphate, sodium hydroxide, said solution having a pH between 7.0 and 7.3; (b) freezing the solution; and (c) lyophilizing the frozen solution.

In another embodiment of the invention, the sterile solution further comprises mannitol.

In another embodiment of the invention, the sterile solution contains pyridoxal 5′-phosphate in a concentration higher than a supplement concentration.

In a further aspect, the present invention provides a kit useful for preparing the injectable formulation according to the invention comprising instructions and in separate containers: (a) the lyophilized formulation of pyridoxal 5′-phosphate of according to the invention; and (b) a sterile carrier suitable for intravenous administration.

In an embodiment of the invention, the kit comprises water for injection as the sterile carrier.

In a further embodiment of the invention, the kit further comprises a container for said injectable formulation, said container sized to faciliate preparation of a selected volume and concentration of said formulation.

In a further aspect, the present invention provides use of a lyophilized formulation according to the invention for the preparation of an injectable formulation suitable for administration to patient in need of treatment with pyridoxal-5-phosphate.

In a further aspect, the present invention provides a method of treating a patient in need of treatment with pyridoxal-5-phosphate comprising intravenously administering the injectable formulation according to the invention

In a further aspect, the present invention provides a method of reducing the incidence of nausea and vomiting associated with the oral administration of pyridoxal 5′-phosphate or a pharmaceutically acceptable salt thereof, said method comprising the step of administering an effective amount of the injectable formulation according to the invention.

In a further aspect, the present invention provides use of an injectable formulation according to the invention for reduction of the incidence of nausea and vomiting associated with the oral administration of pyridoxal 5′-phosphate or a pharmaceutically acceptable salt thereof.

In a further aspect, the present invention provides a method of treating a patient undergoing a surgical procedure in need of treatment with pyridoxal-5-phosphate comprising intravenously administering the injectable formulation according to the invention.

In a further aspect, the present invention provides use of a lyophilized formulation according to the invention for the preparation of an injectable formulation suitable for administration to patient undergoing a surgical procedure in need of treatment with pyridoxal-5-phosphate.

In an embodiment of the invention, the surgical procedure is coronary artery bypass grafting (CABG) or percutaneous coronary intervention (PCI).

### BRIEF DESCRIPTION OF THE FIGURES

FIG. 1 is a line graph comparing the mean measured plasma pyridoxal 5′-phosphate concentration versus time for single intravenous dose of 1, 5, 10, and 20 mg/kg of plasma pyridoxal 5′-phosphate.

FIG. 2 is a line graph comparing the mean baseline adjusted plasma pyridoxal 5′-phosphate concentration versus time for single intravenous dose of 1, 5, 10, and 20 mg/kg of plasma pyridoxal 5′-phosphate.

FIG. 3 is a line graph comparing the mean baseline adjusted plasma pyridoxal 5′-phosphate log concentration versus time for single intravenous dose of 1, 5, 10, and 20 mg/kg of plasma pyridoxal 5′-phosphate.

FIG. 4 is a line graph comparing the mean measured plasma pyridoxal concentration versus time for single intravenous dose of 1, 5, 10, and 20 mg/kg of plasma pyridoxal 5′-phosphate.

FIG. 5 is a line graph comparing the mean baseline adjusted plasma pyridoxal concentration versus time for single intravenous dose of 1, 5, 10, and 20 mg/kg of plasma pyridoxal 5′-phosphate.

FIG. 6 is a line graph comparing the mean baseline adjusted plasma pyridoxal log concentration versus time for single intravenous dose of 1, 5, 10, and 20 mg/kg of plasma pyridoxal 5′-phosphate.

FIG. 7 is a line graph comparing the mean measured plasma 4-pyridoxic acid concentration versus time for single intravenous dose of 1, 5, 10, and 20 mg/kg of plasma pyridoxal 5′-phosphate.

FIG. 8 is a line graph comparing the mean baseline adjusted plasma 4-pyridoxic acid concentration versus time for single intravenous dose of 1, 5, 10, and 20 mg/kg of plasma pyridoxal 5′-phosphate.

FIG. 9 is a line graph comparing the mean baseline adjusted plasma 4-pyridoxic acid log concentration versus time for single intravenous dose of 1, 5, 10, and 20 mg/kg of plasma pyridoxal 5′-phosphate.

FIG. 10 is a line graph comparing the baseline corrected plasma pyridoxal 5′-phosphate mean concentration versus time as measured on Day 1 and on Day 4.

FIG. 11 is a line graph comparing the baseline corrected plasma pyridoxal 5′-phosphate In transformed mean concentration versus time as measured on Day 1 and on Day 4.

FIG. 12 is a line graph comparing the uncorrected plasma pyridoxal 5′-phosphate mean concentration versus time as measured on Day 1 and on Day 4.

FIG. 13 is a line graph comparing the uncorrected plasma pyridoxal 5′-phosphate in transformed mean concentration versus time as measured on Day 1 and on Day 4.

FIG. 14 is a line graph comparing the plasma pyridoxal mean concentration versus time as measured on Day 1 and on Day 4.

FIG. 15 is a line graph comparing the plasma 4-pyridoxic acid in transformed mean concentration versus time as measured on Day 1 and on Day 4.

FIG. 16 is a line graph comparing the plasma 4-pyridoxic acid mean concentration versus time as measured on Day 1 and on Day 4.

FIG. 17 is a line graph comparing the plasma pyridoxal in transformed mean concentration versus time as measured on Day 1 and on Day 4.

### DETAILED DESCRIPTION

The present invention provides novel lyophilized and Injectable formulations of pyridoxal 5′-phosphate and uses thereof. The injectable formulations of pyridoxal 5′-phosphate are suitable for intravenous administration to patients in need of treatment with pyridoxal 5′-phosphate, The injectable formulations of the present invention have improved pharmacokinetics without any significant side effects. Intravenous administration of the injectable formulations of the present invention provides increased sustained plasma levels of pyridoxal 5′-phosphate as compared to prior art formulations.

Previous attempts to prepare a stable intravenous formulation of pyridoxal 5′-phosphate have required the addition of preservatives which were either potentially toxic or potentially carcinogenic. In the absence of preservatives, aqueous solutions of pyridoxal 5′-phosphate degrade rapidly and thus are unsuitable for intravenous administration. The present inventor has found novel lyophilized formulations of pyridoxal 5′-phosphate which can be reconstituted to prepare a stable injectable formulation which does not require the addition of any conventional preservative. Surprisingly, the stability of an intravenous formulation of pyridoxal 5′-phosphate is significantly increased, by lyophilizing the pyridoxal 5′-phosphate with mannitol prior to reconstitution in a suitable carrier. Furthermore, the intravenous formulations so prepared exhibit improved stability without compromising the phermacokinetic properties of its active ingredient, and without any significant side effects.

The formulations of the present invention are particularly useful for the treatment of cardiovascular and cerebrovascular complications. Coronary artery bypass grafting (CABG) and percutaneous coronary intervention (PCI) are two of the most frequently performed procedures in North America. During the surgeries patients are infused with antiplatelet and antithrombotic therapies to reduce the frequency of ischemic complications. Specifically, the therapies used are heparin, abciximab and eptifibatide. They can be used alone or in combination with each other. The present invention provides formulations of pyridoxal 5′-phosphate which can be administered intravenously during CABG or PCI to further reduce ischemic complications.

The formulations according to the invention are also suitable for administration to stroke patients unable to swallow an oral form of pyridoxal 5′-phosphate such as a tablet. As well, in a hospital setting, an intravenous formulation of pyridoxal 5′-phosphate, as opposed to an oral formulation, provides the advantages of providing higher plasma levels of pyridoxal 5′-phosphate without inducing the gastric irritation, nausea, vomiting and diarrhea which can be associated with oral administration of pyridoxal 5′-phosphate. In addition, intravenous formulations achieve high plasma levels within minutes whereas an oral formulation may require hours to reach similar levels. This makes the intravenous formulation ideal for emergency situations (for example, treatment of MI or stroke, emergency PCI or bypass) in which oral administration is not possible or ideal.

### Lyophilized Formulation of Pyridoxal 5′-Phosphate and Method of Preparation

In one aspect, the present invention provides a lyophilized formulation of pyridoxal 5′-phosphate having been prepared by lyophilizing a frozen sterile aqueous solution of pyridoxal 5′-phosphate, sodium hydroxide and optionally, mannitol.

The lyophilized formulation of pyridoxal 5′-phosphate comprises a lyophilized sterile aqueous solution of pyridoxal 5′-phosphate in a concentration higher than a supplement concentration.

“Supplement concentration” as used herein is defined to mean a concentration which is intended for restoring a vitamin or metabolite level to a normal level in a person suffering from a vitamin or metabolite deficiency, and excludes a concentration which is suitable for increasing a vitamin or metabolite level to an above normal metabolic level.

The lyophilized formulation can be prepared using pyridoxal 5′-phosphate or a pharmaceutically acceptable salt thereof. Both the monohydrate and the anhydrous forms of pyridoxal 5′-phosphate are suitable for preparation of the pharmaceutical compositions of the invention. The pyridoxal 5′-phosphate may be provided as salt forms with pharmaceutically compatible counterions such as but not limited, to citrate, tartate, bisulfate, etc. The pharmaceutically compatible salts may be formed with many acids, including but, not limited to, hydrochloric, sulfuric, acetic, lactic, tartaric, malic, succinic, etc. The salt forms tend to be more soluble in aqueous or other protonic solvents than the corresponding free base forms.

A first step in preparing the lyophilized formulation of pyridoxal 5′-phosphate is to prepare a sterile solution comprising pyridoxal 5′-phosphate and sodium hydroxide.

In preferred embodiment, the sterile solution has a pH of about between 7.0 and 7.3. The solution is prepared by first dissolving the pyridoxal 5′-phosphate and sodium hydroxide in a suitable amount of water and adjusting the pH.

The term “percentage weight per weight (% w/w)” as used herein refers to the weight percentage of the particular compound or carrier relative to the total weight of the composition of which the compound or carrier is a constituent of.

In an embodiment, the sterile solution will contain about 1 to 25% w/w pyridoxal 5′-phosphate.

In a preferred embodiment, the sterile solution will contain about 1 to 15% w/w pyridoxal 5′-phosphate.

In a further preferred embodiment, the sterile solution will contain about 1 to 10% w/w pyridoxal 5′-phosphate.

In a still further preferred embodiment, the sterile solution will contain about 5% w/w pyridoxal 5′-phosphate.

In another embodiment, the sterile solution will contain about 0.1 to 10% w/w sodium hydroxide.

In a preferred embodiment, the sterile solution will contain about 0.5 to 5% w/w sodium hydroxide.

In a further preferred embodiment, the sterile solution will contain about 0.5 to 3% w/w sodium hydroxide.

In a still further preferred embodiment, the sterile solution will contain about 1.5% w/w sodium hydroxide.

In another embodiment, the sterile solution may further comprise mannitol. In embodiments where the sterile solution includes mannitol, the sterile solution is prepared by first dissolving the pyridoxal 5′-phosphate and sodium hydroxide in a suitable amount of water and adjusting the pH, followed by dissolving the mannitol in the pyridoxal 5′-phosphate/sodium hydroxide solution. The resulting solution is then sterilized, for example, by filter sterilization.

In one embodiment, the sterile solution will contain about 0.2 to 10% w/w mannitol.

In one embodiment, the sterile solution will contain about 0.5 to 5% w/w mannitol.

In a preferred embodiment, the sterile solution will contain about 3% w/w mannitol.

In a more preferred embodiment, the sterile solution will contain about 5% w/w of pyridoxal 5′-phosphate, about 1.5% w/w of sodium hydroxide and about 2.8% w/w of mannitol.

The sterilize solution may then be dispensed into sterile plastic or glass containers such as ampoules or vials in suitable volumes.

The amount of pyridoxal 5′-phosphate packaged into a single vial may vary between 25 mg and 1000 mg, between 50 and 1000 mg and more preferably between 100 and 750 mg. The amount of the sodium hydroxide and mannitol will depend on the amount of pyridoxal 5′-phosphate. In one embodiment of the invention, a single unit dosage form of the lyophilized formulation comprises about 250 mg of pyridoxal 5′-phosphate, 80.5 mg of sodium hydroxide, 150 mg of mannitol and 5.0 ml WFI (water for injection).

Once the sterile solution has been aliquoted into containers, the solution is frozen at temperature of between −20 and −45° C. The substantially frozen aqueous solution may be maintained at this temperature until lyophilization is commenced.

Lyophilization of the substantially frozen aqueous solution may be carried out involving, for example, both primary drying and secondary drying. Primary drying may be carried out via sublimation by using controlled application of vacuum and heat, for example under a substantial vacuum of about 0.1 to 0.5 Torr for sufficient time to effect removal of substantially all the frozen water and/or other solvent. Secondary drying is preferably carried out subsequently under a substantially similar vacuum to remove as much as possible of the last traces of adsorbed water or other solvent, thus providing a dry cake or powder.

The temperature at which primary drying is carried out ranges from −10 to 0° C. at the beginning of the process so as to maintain the solution in a substantially or completely frozen form. As the process proceeds and the product temperature reaches the desired shelf temperature, the primary drying phase is completed. The temperature at which secondary drying is carried out ranges from 25 to 35° C. in order to remove any adsorbed water and/or other solvent. The moisture content of the resulting cake or powder is preferably less than 2.5% by weight. Once the lyophilization has been completed, the sterile, plastic or glass container containing the lyophilized formulation may then be stoppered or sealed.

The resulting lyophilized formulation is physically and chemically stable when stored in low light conditions and at temperatures between 2 to 8° C.

### Injectable Formulation of Pyridoxal 5′-Phosphate and Method of Preparation

In another aspect, the present invention provides an injectable formulation containing pyridoxal 5′-phosphate in a concentration higher than a supplement concentration, reconstituted from a lyophilized formulation according to the invention, using a sterile carrier suitable for intravenous administration. Preferably, the lyophilized formulation according to the invention is reconstituted in an appropriate volume of WFI (water for injection) to provide the injectable formulation. The resulting injectable formulation is particularly suitable for bolus administration. Preferably, the concentration of pyridoxal 5′-phosphate in the injectable formulation will be in the range of 1 to 100 mg/ml, 5 to 75 mg/ml, and more preferably between 10 and 50 mg/ml.

### Kits for the Preparation of an Injectable Formulation of Pyridoxal 5′-Phosphate

In a further aspect, the present invention provides a kit useful for preparing the injectable formulation according to the invention comprising instructions for preparing the injectable formulation and in separate containers: (a) the lyophilized formulation of pyridoxal 5′-phosphate of according to the invention; and (b) a sterile carrier suitable for intravenous administration. In a preferred embodiment, the sterile carrier is WFI. In another preferred embodiment, the kit further comprises a container for the injectable formulation, said container sized to faciliate preparation of a selected volume and concentration of the injectable formulation.

The lyophilized formulation and the sterilized carrier can be aliquoted into volumes suitable for a single bolus injection. The lyophilized formulation and the sterilized carrier can be aliquoted into volumes suitable for extended administration. The kit can comprise a container, the container being sized to facilitate preparation of the formulation for injection to specified concentrations and volumes, without further measuring. The container can be a syringe body or suitable for use with a syringe.

### Uses of Injectable Formulation of Pyridoxal 5′-Phosphate

A limiting factor in the tolerance to high doses of pyridoxal 5′-phosphate is gastrointestinal discomfort characterized mainly by nausea and vomiting. The present invention provides novel injectable formulations suitable for the intravenous administration of high doses of pyridoxal 5′-phosphate with minimal gastrointestinal side effects associated with oral administration. The injectable formulations according to the invention overcome the stability issues of prior art intravenous formulations of pyridoxal 5′-phosphate. The stability of aqueous solutions of pyridoxal 5′-phosphate contained in the injectable formulations of the present invention are improved over the prior art pyridoxal 5′-phosphate formulations which are highly light sensitive in solution, and to a lesser degree in powder form. The intravenous formulations of according to the invention do not require or require to a lesser degree, the inclusion of preservatives for improving stability and for increasing shelf life, as compared to the prior art formulations.

Thus a method of treating a patient in need of treatment with pyridoxal-5-phosphate is provided comprising intravenously administering the injectable formulation according to the invention.

In another aspect, provided is a method of reducing the incidence of nausea and vomiting associated with the oral administration of pyridoxal 5′-phosphate or a pharmaceutically acceptable salt thereof, said method comprising the step of administering an effective amount of the injectable formulation according to the invention.

Intravenous administration of pyridoxal-5′-phosphate is particularly useful for treatment of patients undergoing a surgical procedure and requiring antiplatelet and antithrombotic therapies to reduce the frequency of ischemic complications. In a further aspect, provided is a method of treating a patient undergoing a surgical procedure in need of treatment with pyridoxal-5-phosphate comprising intravenously administering the injectable formulation according to the invention. The surgical procedure may be a cardiovascular related procedure such as coronary artery bypass grafting (CABG) or percutaneous coronary intervention (PCI).

An individual dose of the injectable formulation may contain between 10 and 1000 mg of pyridoxal-5′-phosphate, preferably between 50 mg and 100 mg, between 100 and 1000 mg of pyridoxal-5′-phosphate and more preferably between 250 and 1000 mg of pyridoxal 5′-phosphate. The injectable formulations according to the invention are suitable for once or twice daily administration, for example such as a single bolus injection. The injectable formulations may also be used for extended or continuous administration.

The injectable formulations generally are administered in an amount effective for treatment or prophylaxis of a specific indication or indications. It is appreciated that optimum dosage will be determined by standard methods for each treatment modality and indication, taking into account the indication, its severity, complicating conditions and the like. A therapeutically effective dose further refers to that amount of the compound sufficient to result in amelioration of symptoms associated with such disorders. Techniques for formulation and administration of the compounds of the instant application may be found in “Remington's Pharmaceutical Sciences,” Mack Publishing Co., Easton, Pa., latest edition. For administration to mammals, and particularly humans, it is expected that the daily dosage level of the active agent will be 100 to 1000 mg, typically around 200 to 500 mg. The physician in any event may determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

Although the invention has been described with reference to illustrative embodiments, it is to be understood that the invention is not limited to these precise embodiments, and that various changes and modifications may be effected therein by one skilled in the art. All such changes and modifications are intended to be encompassed in the appended claims.

### EXAMPLES

### Example One

### Preparation of a Lyophilized Formulation of Pyridoxal 5′-Phosphate (P5P)

Table 1 sets outs the formulation for individual doses of the lyophilized formulation of pyridoxal 5′-phosphate. Each vial provides 250 mg of P5P.

TABLE 1
Formulation

| Ingredient | Quantity per vial | |
|---|---|---|
| Sodium Hydroxide | 80.5 | mg |
| P5P | 250 | mg |
| Mannitol | 150 | mg |
| WFI (water for injection) | 5.0 | ml |

The following procedure is for the preparation of a 5 L batch of the lyophilized formulation of pyridoxal 5′-phosphate. The batch size can be scaled up or down by increasing or decreasing the relative amounts proportionately.

Preparation of Glassware and Vials—Glass vials were washed and depyrogenated at a temperature of 250° C.±15° C. All glassware and apparatus used in the preparation of the lyophilized formulation including magnetic stir bars, pipettes, Erlenmeyer flasks, flasks, filters, stoppers, and seals, were sterilized by autoclaving at a temperature of 121° C.±3° C. for 45 minutes.

Preparation of P5P Solution—In calculating the amount of P5P required to make a final concentration of 250 mg per vial, a correction factor of 1.08 was used as the water content of the active pharmaceutical ingredient is 7.71%.

For each 5 L batch, 4 kg of WFI (80% of total required) was nitrogen flushed in the core of the solution. With magnetic stirring throughout, 80.6 g of sodium hydroxide and 270.4 g of P5P were dissolved in the 4 kg of WFI to provide a clear solution. The pH of the solution was adjusted accordingly with either 1N NaOH or 1N HCl to a pH of 7.0 to 7.3. Following pH adjustment, 150.3 mg of mannitol was dissolved in the P5P/NaOH solution with continuous magnetic stirring until the solution was clear. Additional WFI was then added to adjust the weight of the solution to 5.245 kg (density 1.049 g/ml). The solution was then sterile filtered through a 0.2 μm Milipack K40 gamma gold model filter and the filter integrity tested.

Lyophilization—Individual vials were sterilely filled in a class 100 clean room using a dispensing pump set for dosage volume range of 5.10-5.30 mL (equivalent to 5.35-5.56 g per vial—by weight). The vials were partially stoppered with bromobuytl stoppers free of 2-mercaptobenzothiazole. Trays of the partially stoppered vials were loaded into lyophilization chambers which had been pre-chilled to 4° C. Lyophilization was preformed using the following parameters: Cooling temperature: −45° C.; Primary cooling time—6 hours; Primary drying time (in nitrogen atmosphere)—50 hours; Secondary drying temperature: 35° C.; Secondary drying time—48 hours.

After lyophilization, the vials were crimped with aluminum seals having an attached plastic button. The vials were stored at 2-8° C.

### Example Two

### Comparison of Bioavailability of Intravenously Versus Orally Administered Pyridoxal 5′-Phosphate (P5P)

A phase one clinical trial was conducted to compare the pharmacokinetics of intravenously administered P5P and orally administered P5P. The trial was a single dose escalation study.

For intravenous administration, P5P was administered by bolus injection under fasting conditions. The following doses of P5P were studied: 1, 5, 10, and 20 mg/kg. 6 subjects were assigned to each dosage group.

For oral administration, P5P was administered as a single non-coated tablet or as a single enteric coated tablet, under fasting conditions. For the non-coated tablets, the following doses of P5P were studied: 5, 10, 17.5, and 25 mg/kg. For the enteric coated tablets, the following doses of P5P were studied: 15, 30 and 60 mg/kg.

P5P, pyridoxal, and 4-pyridoxic acid were evaluated in both plasma and urine over a 24 hour interval following either intravenous or oral dosing. The results are set out below in Tables 2 and 3.

TABLE 2
Pharmacokinetic Parameters for Single Intravenous Dose of P5P

| Dose | Tmax | T½ | Cmax | AUC-T |
|---|---|---|---|---|
| (mg/kg) | (h) | (h) | (μg/ml) | (μg · h/ml) |
| 1 | 0.14 ± 0.07 | 5.11 ± 0.98 | 19.97 ± 2.95 | 38.41 ± 4.61 |
| 5 | 0.14 ± 0.07 | 5.89 ± 0.39 | 98.82 ± 16.03 | 172.85 ± 31.95 |
| 10 | 0.10 ± 0.03 | 5.87 ± 0.89 | 219.98 ± 9.52 | 307.97 ± 45.48 |
| 20 | 0.15 ± 0.07 | 5.03 ± 0.72 | 424.06 ± 96.88 | 577.09 ± 62.45 |

TABLE 3
Pharmacokinetic Parameters for Single Oral Dose of P5P

| | Dose | Tmax | T½ | Cmax | AUC-T |
|---|---|---|---|---|---|
| | (mg/kg) | (h) | (h) | (μg/ml) | (μg · h/ml) |
| Non-coated | 5 | 9.2 ± 7.7 | 46 ± 27 | 0.10 ± 0.03 | 2.76 ± 1.22 |
| tablet | 10 | 6.6 ± 6.5 | 47 ± 11 | 0.10 ± 0.02 | 3.31 ± 1.22 |
| | 17.5 | 0.5 ± 0.0 | 54 ± 15 | 0.27 ± 0.08 | 2.54 ± 1.06 |
| | 25 | 12.9 ± 5.8 | 52 ± 32 | 0.09 ± 0.02 | 3.93 ± 2.25 |
| Enteric Coated | 15 | 7.0 ± 2.8 | 10.0 ± 4.9 | 2.84 ± 2.99 | 12.04 ± 10.49 |
| Tablet | 30 | 4.3 ± 1.2 | 20.2 ± 15.6 | 6.43 ± 12.42 | 25.80 ± 45.08 |
| | 60 | 4.5 ± 0.5 | 15.3 ± 6.3 | 4.47 ± 5.10 | 18.69 ± 17.83 |

The results indicate that the bioavailability of P5P was greater when intravenously administered rather than orally administered.

### Example Three

### Comparison of Pharmacokinetics and Incidence of Adverse Events for Single Intravenous Doses of 1, 5, 10, and 20 mg/kg of Pyridoxal 5′-Phosphate (P5P)

Methodology—This was a single-blind, single-dose escalation, non-randomized, 4-phase, non-crossover study designed to evaluate the safety and tolerability of P5P intravenous injectable solution at doses of 1, 5, 10, 20 mg/kg, administered to four groups of healthy male and female subjects under fasting conditions and to estimate pharmacokinetic parameters of P5P.

Subjects were assigned to one of the four groups (six subjects per group) under fasting conditions.

Concentrations of pyridoxal-5′-phosphate (P5P) were measured from the plasma samples collected over a 24-bour interval after dosing in each phase.

Pharmacokinetic parameters: AUCₜ, AUC_{inf}, Cₘₐₓ, Tₘₐₓ, Kₑₗ, T_{half}, V_{d}, CL, MRT and F were estimated based on pyridoxal-5′-phosphate (P5P) plasma levels for each subject that was in the final data set.

Safety data were collected for each subject throughout the study by recording vital signs, ECGs and reported adverse events.

### Number of Subjects (Planned and Analyzed)

Six (6) subjects were dosed in Group 1 (1 mg/kg). Six (6) subjects completed phase I of the study and were analyzed.

Six (6) subjects were dosed in Group 2 (5 mg/kg). Six (6) subjects completed phase 2 of the study and were analyzed.

Six (6) subjects were dosed in Group 3 (10 mg/kg). Six (6) subjects completed phase 3 of the study and were analyzed.

Six (6) subjects were dosed in Group 4 (20 mg/kg). Five (5) subjects completed phase 4 of the study and were analyzed.

Diagnosis and Main Criteria for Inclusion

Subjects met all of the following inclusion criteria within 21 days prior to drug administration.

1) Healthy, non-smoking, male and female subjects, 18 to 55 years of age (inclusive).

2) Body weight within ±10% of the appropriate weight for the subject's height and frame (as published in the 1983 Metropolitan Life Insurance Company Scale, Statistical Bureau).

3) Negative for: HIV; Hepatitis B surface antigen and Hepatitis C antibody; Urine tests for drugs of abuse (marijuana, amphetamines, barbiturates, cocaine, opiates. benzodiazepines and methadone); Cotinine (urine test); Serum HCG (females only)

4) No significant diseases or clinically significant findings in a physical examination.

5) No clinically significant abnormal laboratory values.

6) No clinically significant findings in vital signs measurements and a 12-lead electrocardiogram (ECG).

7) Be informed of the nature of the study and given written consent prior to receiving any study procedure.

8) Females who participate in this study are: unable to have children (e.g. post-menopausal, tubal ligation, hysterectomy) OR willing to remain abstinent [not engage in sexual intercourse] OR willing to use an effective method of double-barrier birth control [partner using condom and female using diaphragm, contraceptive sponge, spermicide or IUD] OR willing to use a hormonal contraceptive [oral, inserted under the skin, patch or injection].

9) Females who participate in this study are non-lactating.

The test product used was a P5P Intravenous Injectable Solution, 50 mg/ml, which was prepared in accordance with the methods of Example 1 (CanAm BioResearch Inc. Canada); Lot No.: LP1459: Manufacturing Date: 12/03. A single 1, 5, 10 or 20 mg/kg dose was given by intravenous injection. The duration of treatment was a single dose.

### Criteria for Evaluation

Primary Endpoint—Safety Parameters—The safety parameters investigated in this study were vital signs measurements (blood pressure, pulse rate), ECG measurements (1, 2 and 12 hours post-dose), platelet function testing, and severity and causality of adverse events experienced by subjects during the study period and laboratory assessments (clinical chemistry, hematology, urinalysis).

Secondary Endpoint—Pharmacokinetic (PK) Parameters—Plasma concentrations of pyridoxal-5′-phosphate (P5P) were measured by a validated analytical method. Based on these concentration levels, AUCₜ, AUC_{inf}, Cₘₐₓ, Tₘₐₓ, Kₑₗ, T_{half}, V_{d}, CL, MRT and F were estimated.

Statistical Methods—Descriptive statistics were calculated by treatments for the estimated pharmacokinetic parameters.

### Discussion and Conclusions

Overall the safety profile of P5P injectable solution established in this clinical study demonstrates that the product is well tolerated with no serious or lasting treatment related effects at doses up to 20 mg/kg, when administered as a single dose. Common side effects of P5P observed in this study include nausea, vomiting and stomach upset. None of the adverse events had a significant impact on the safety of the subjects or integrity of the study results.

Based on the adverse event profile generated from the results of this study, P5P is safe and well tolerated in doses up to 20 mg/kg.

The P5P pre-dose concentrations were very low, less than 1% of the corresponding Cₘₐₓ values (range: 0.01% to 0.44%). The pyridxoal (PAL) pre-dose concentrations were slightly larger, between 0.06% and 2.47% of the Cₘₐₓ values.

Only one subject (Subject 23) exhibited a non-zero pre-dose level for 4-pyridoxic acid (PA) and it represented 0.06% of the Cₘₐₓ parameter. Note that where applicable, the baseline (mean value of the pre-dose levels) were subtracted from the measured analyte levels before the pharmacokinetic analysis of the data.

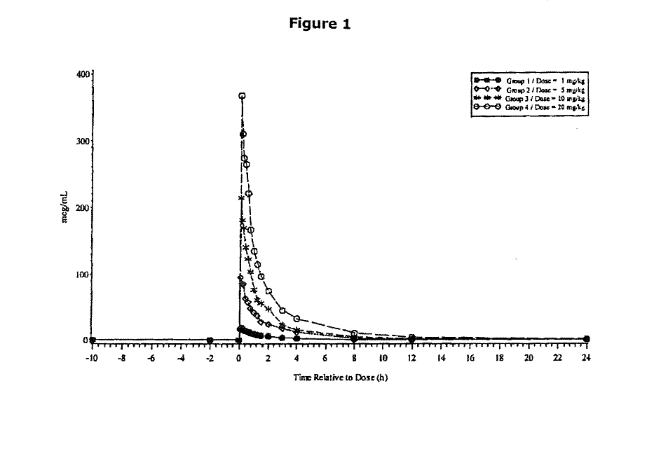

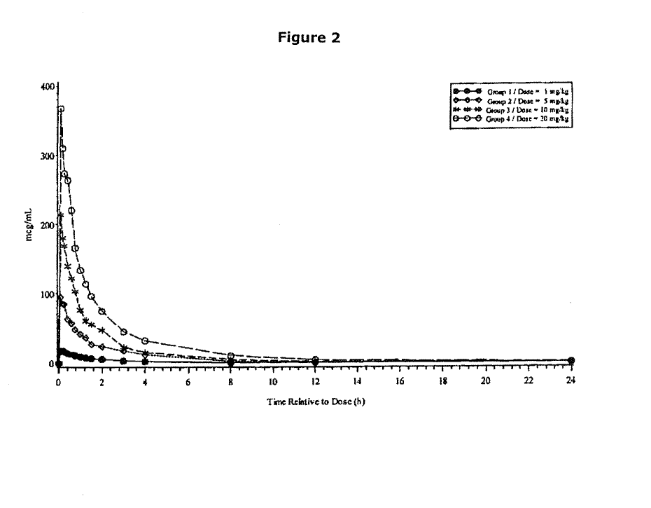

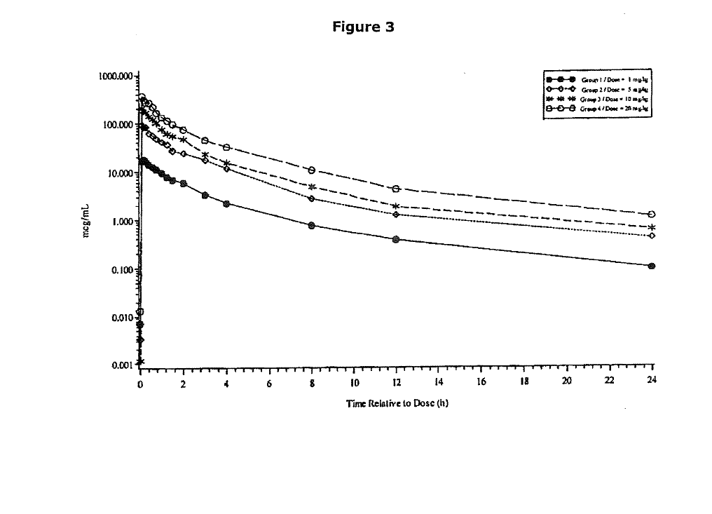

The P5P concentration-time profiles after the initial rapid increase exhibited a moderate decrease, attributable mainly to a distribution phase followed by a slower elimination phase (FIGS. 1 to 3). Only 3 data-points were considered on the terminal linear phase and used for the estimation of the apparent elimination half-life. It is possible that the 24-hour sampling interval was not long enough to capture the real elimination phase of P5P. The estimated values were consistent over all doses, between 5 and 6 hours (Table 4).

TABLE 4
Plasma Pharmacokinetic Parameters for P5P
Plasma Pharmacokinetic Parameters for Pyridoxal-5′-Phospbate (P5P)
Mean (CV %)

| | | Group | | | | | | | |
|---|---|---|---|---|---|---|---|---|---|
| | | 1 | | 2 | | 3 | | 4 | |
| | | Dose of P5P | | | | | | | |
| | | 1 mg/kg | | 5 mg/kg | | 10 mg/kg | | 10 mg/kg | |
| | | No. of subjects | | | | | | | |
| | | 6 | | 6 | | 6 | | 5 | |
| AUC (O-t) | (μ · g * ′h/mL) | 38.413 | (12) | 172.849 | (18) | 301.913 | (15) | 577.091 | (11) |
| AUC (o-inf) | (μ · g * h/mL) | 39.116 | (13) | 175.910 | (19) | 312.439 | (15) | 584.564 | (11) |
| Cmax | (μg/mL) | 19.968 | (15) | 98.823 | (16) | 219.930 | (4) | 424.058 | (23) |
| Tmax | (h) | 0.14 | (49) | 0.14 | (49) | 0.10 | (35) | 0.15 | (47) |
| Kel | (l/h) | 0.1399 | (10) | 0.1180 | (7) | 0.1205 | (16) | 0.1400 | (15) |
| T½ | (h) | 5.11 | (19) | 5.89 | (1) | 5.81 | (15) | 5.03 | (14) |
| MRT | (h) | 3.73 | (27) | 3.52 | (12) | 3.11 | (12) | 3.32 | (11) |
| CL | (mL/h) | 1759 | (16) | 1928 | (23) | 21110 | (24) | 2385 | (18) |
| Vd | (mL) | 12959 | (22) | 16336 | (22) | 18002 | (13) | 17543 | (30) |

The distribution dominated phase was not clearly observable for the two metabolites, pyridoxal (PAL) and 4-pyridoxic acid (PA). The apparent elimination half-life was approximately 4 hours for PAL (Table 5) and approximately 2 to 3.5 hours for PA (Table 6).

TABLE 5
Plasma Pharmacokinetic Parameters for PAL
Plasma Pharmacokinetic Parameters for Pyridoxal (PAL)
Mean (CV %.)

| | | Group | | | | | | | |
|---|---|---|---|---|---|---|---|---|---|
| | | 1 | | 2 | | 3 | | 4 | |
| | | Dose of P5P | | | | | | | |
| | | 1 mg/kg | | 5 mg/kg | | 10 mg/kg: | | 20 mg/kg | |
| | | No. of subjects | | | | | | | |
| | | 6 | | 6 | | 6 | | 5 | |
| AUC (0-t) | (μ * g · h/mL) | 2.020 | (34) | 10.132 | (13) | 39.143 | (16) | 94.403 | (20) |
| AUC (0-inf) | (μg * h/lmL) | 2.070 | (32) | 10.365 | (14) | 39.488 | (16) | 95.185 | (20) |
| Cmax | (μ/lmL) | 0.591 | (26) | 2.480 | (18) | 13.267 | (23) | 25.902 | (13) |
| Tmax | (h) | 0.46 | (54) | 0.71 | (92) | 0.42 | (36) | 0.32 | (29) |
| Kel | (l/h) | 0.1980 | (37) | 0.1797 | (30) | 0.1666 | (11) | 0.1770 | (18) |
| T½ | (h) | 3.96 | (40) | 4.25 | (39) | 4.20 | (11) | 4.01 | (16) |
| MRT | (h) | 3.70 | (16) | 4.40 | (19) | 3.68 | (13) | 3.98 | (8) |

TABLE 6
Plasma Pharmacokinetic Parameters for PA
Plasma Pharmacokinetic Parameters for 4-Pyridoxic Acid (PA)
Mean (CV %)

| | | Group | | | | | | | |
|---|---|---|---|---|---|---|---|---|---|
| | | 1 | | 2 | | 3 | | 4 | |
| | | Dose of P5P | | | | | | | |
| | | 1 mg/kg | | 5 mg/kg: | | 10 mg/kg | | 20 mg/kg | |
| | | No. of subjects | | | | | | | |
| | | 6 | | 6 | | 6 | | 5 | |
| AUC(0-t) | (μg * h/mL) | 1.504 | (55) | 10.482 | (14) | 22.666 | (16) | 51.901 | (23) |
| AUC(0-inf) | (μg * h/mL) | 1.993 | (34) | 10.749 | (14) | 22.982 | (15) | 53.021 | (14) |
| Cmax | (μg/mL)) | 0.419 | (32) | 2.447 | (15) | 5.361 | (27) | 16.065 | (21) |
| Tmax | (h) | 1.04 | (24) | 0.71 | (23) | 0.74 | (53) | 0.73 | (51) |
| Kel | (l/h) | 0.311 | (53) | 0.3315 | (13) | 0.2676 | (24) | 0.2158 | (32) |
| T½ | (h) | 2.82 | (52) | 2.12 | (13) | 2.73 | (21) | 3.48 | (31) |
| MRT | (h) | 4.50 | (45) | 3.48 | (8) | 3.85 | (7) | 4.13 | (15) |

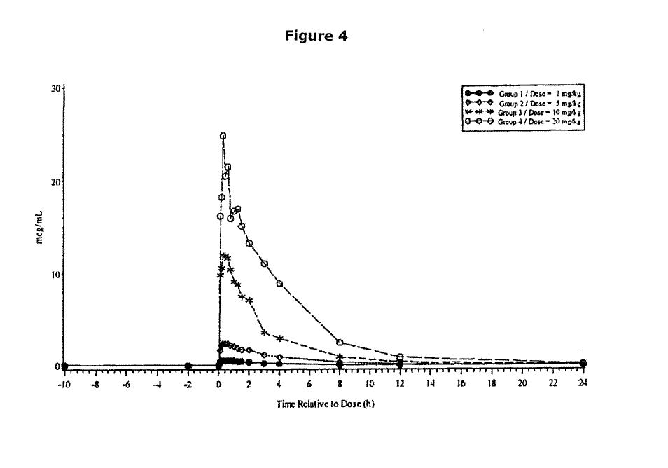

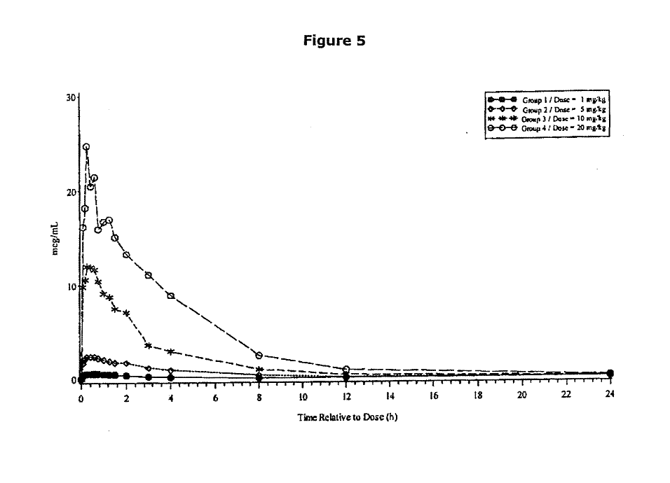

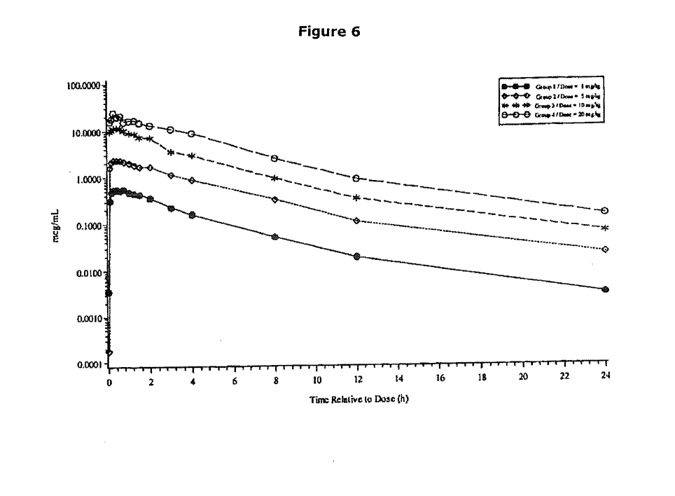

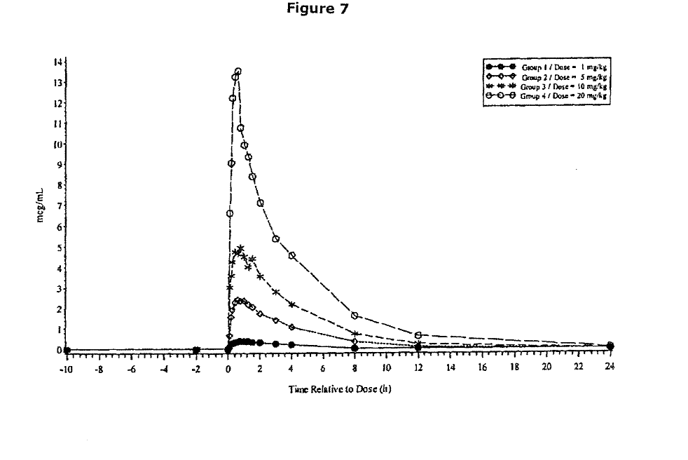

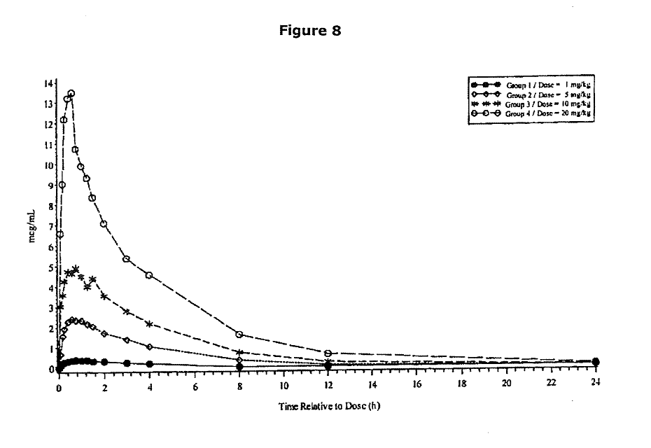

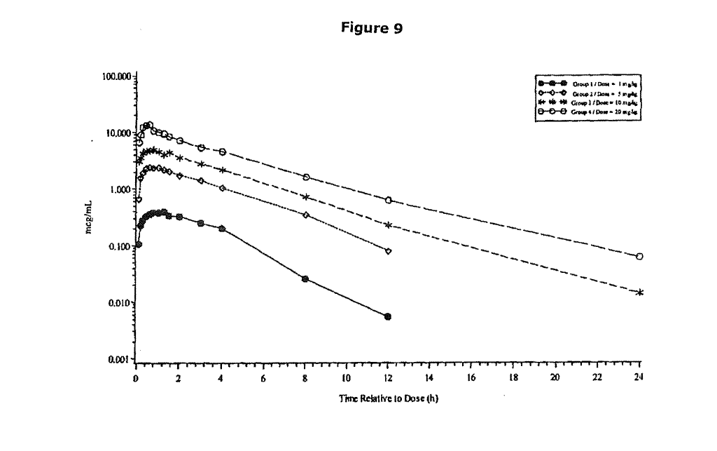

PAL reached maximum plasma levels at approximately 0.30 to 0.70 hours (FIGS. 4 to 6), while PA attained maximum concentrations at approximately 0.70 to 1.0 hours (FIGS. 7 to 9), after the beginning of the iv. infusion. These values are consistent with the sequential formation of these metabolites.

The urinary excretion of the unchanged P5P is negligible. Less than 0.5% of the administered dose was found as P5P in the urine (Table 10). Similarly, very little of the dose was excreted in urine as PAL, between approximately 2 to 3% of the total dose (Table 10).

PA represented the largest amounts found in urine. Over a 24-hour interval, between 30% and 52% of the given dose was excreted in urine as 4-pyridoxic acid.

The results of the analysis of the relationship between the values of pharmacokinetic parameters and dose are summarized in the Table 7 below.

TABLE 7
Summary of Urinary Excretion
Cumulative Urinary Excretion as Percent of the Dose
Percent (CV %)

| | Group | | | |
|---|---|---|---|---|
| | 1 | 2 | 3 | 4 |
| Dose of P5P | 1 mg/1 kg | 5 mg/kg | 10 mg/kg | 26 mg/kg |
| No. of | 6 | 6 | 6 | 5 |
| subjects | | | | |
| P5P | 0.06 (161) | 0.08 (96) | 0.02 (167) | 0.26 (73) |
| PAL | 1.93 (101) | 3.00 (37) | 2.73 (20) | 2.46 (33) |
| PA | 52.25 (28) | 47.18 (32) | 29.78 (32) | 40.55 (54) |
| Total | 54.24 (27) | 50.26 (30) | 32.53 (30) | 43.27 (52) |

TABLE 8
Summary of Pharmacokinetic Parameters versus Dose
Relationship Equation
Summary of PK Parameters versus Dose Relationship
Equation: ln(pK Parameter) = b0 + b1 * ln (Dose)

| | | Coef. | 95% | 95% | | |
|---|---|---|---|---|---|---|
| | | for | Lower | Upper | | |
| | Intercept | Dose | Limit | Limit | Significance | |
| Parameter | (b0) | (b1) | (b1) | (b1) | of b1 | R² |
| P5P | | | | | | |
| lnAUCi | 3.67 | 0.90 | 0.85 | 0.96 | <0.0001 | 0.98107 |
| lnCmax | 2.98 | 1.03 | 0.97 | 1.08 | <0.0001 | 0.98411 |
| lnCl | 7.44 | 0.10 | 0.02 | 0.17 | 0.0128 | 0.26048 |
| lnThalf | 1.67 | 0.02 | −0.05 | 0.08 | 0.6177 | 0.01207 |
| lnMRT | 1.29 | −0.05 | −0.11 | 0.02 | 0.1568 | 0.09314 |
| PAL | | | | | | |
| lnAUCi | 0.56 | 1.30 | 1.18 | 1.41 | <0.0001 | 0.96365 |
| lnCmax | −0.72 | 1.30 | 1.16 | 1.45 | <0.0001 | 0.94194 |
| lnThalf | 1.33 | 0.03 | 0.08 | 0.14 | 0.5604 | 0.01640 |
| lnMRT | 1.33 | 0.02 | −0.05 | 0.08 | 0.5671 | 0.01585 |
| PA | | | | | | |
| lnAUCi | 0.62 | 1.10 | 1.01 | 1.18 | <0.0001 | 0.97052 |
| lnCmax | −0.98 | 1.20 | 1.09 | 1.31 | <0.0001 | 0.96098 |
| lnThalf | 0.82 | 0.08 | −.006 | 0.22 | 0.2604 | 0.0599 |
| lnMRT | 1.37 | −0.01 | −0.11 | 0.09 | 0.7995 | 0.00314 |

Both plasma AUC_{inf} and Cₘₐₓ parameters are dose-dependent for all analytes. The coefficient of determination (R²) for the linear regression is quite high and the estimated coefficient for the dose (b1) is significantly different from zero.

The dose-proportionality is clearly demonstrated for plasma P5P Cₘₐₓ parameter since the 95% confidence interval (CI) of b1 contains 1. In the case of plasma AUC_{inf} of P5P appears to be a slight deviation from proportionality. The 95% CI for b1, 0.85-0.96, does not contain the value of 1. However, the deviation seems quite small and it may be explained by the experimental error. This deviation may be the reason for the dose dependency observed for the clearance, CL. The b1 coefficient is significantly different from zero but the coefficient of determination is very low, 0.26048, and the value of b1 is very low as well, 0.10.

In the case of the metabolites, PAL and PA, both parameters, AUC_{inf} and Cₘₐₓ, seem to increase more than expected based on dose. The 95% confidence intervals for the b1 coefficient lay above the value of 1. This deviation is more pronounced for the PAL analyte.

The apparent elimination half-life (T_{half}) and the mean residence time (MRT) are independent of the dose for all 3 analytes. The coefficient of the dose (b1) is not significantly different from zero, therefore it may be concluded that the elimination half-life and the mean residence time are constant over the 1 mg/kg to 20 mg/kg dose range.

### Example Four

### Bioavailability and Incidence of Adverse Events for Single Intravenous Dose of 10 mg/kg of Pyridoxal 5′-Phosphate (P5P) Over 4 Day Period

Data sets analyzed—Six (6) healthy, adult non-smokers were administered intravenously a single daily dose of 10 mg/kb of P5P for four consecutive days and completed the study. In accordance with the study protocol, data from all subjects who completed the study were used for pharmacokinetic and statistical analysis.

Demographics—Age (years): mean=33±10; range=19-49; median=32. Height (cm): mean=168.7±4.8; range=163.0-175.5; median=68.75. Weight (kg): mean=70.9±10.0; range=56-0-83.2; median=68.75. BMI (kg/m²): mean=24.9±3.0; range=20.6-29.3; median=25.

Analytical Methods—Plasma and urine samples were used for analysis. P5P, PAL, and PA levels were determined by HPLC/FLD. The quantitation level was 5 ng/mL for plasma and 100 ng/mL for urine. The sample analysis calibration curve range was 5 ng/mL to 5000 ng/mL for plasma and 100 ng/mL to 50,000 ng/mL for urine.

Safety Methods—The safety parameters investigated in this study were: adverse events, vital sign measurements, ECGs, physical examination and standard laboratory evaluations.

### Measurements of Treatment Compliance

Measurements of treatment compliance were 100% as subjects were dosed under direct observation; subject identification was verified and cross-checked with the pre-dispensed medication. In addition, subjects were confined to the SFBC Anapharm Clinical Research Facility from at least 10 hours prior to placebo injection, on Day 0 until after the 24.0-hour post 4th dose blood draw on Day 5.

### Statistical Methods

The P5P, PAL and PA plasma concentration data for each subject, sampling time and treatment day were electronically transferred from BRI Biopharmaceutical Research Inc. to the statistical division of SFBC An.apbam1. The data were cross-checked against the hard copies.

Time deviations during sampling were treated as follows: for all sampling times, the difference between the scheduled and the actual sampling time was considered acceptable if it was inferior to 1 minute. When the difference exceeded this time limit, the actual sampling times (rounded off to three decimal digits) were used to calculate pharmacokinetic parameters except for pre-dose samples, which were always reported as zero (0.000), regardless of time deviations. Scheduled sampling times are presented in concentration tables and graphs of the statistical section of the report.

Samples that were not available were recorded as NS (No sample) in the concentration tables. These samples were treated as missing for pharmacokinetic and statistical analyses.

Pharmacokinetic analyses (non-compartmental and compartmental) were performed at SFBC Anapharm. Pharmacokinetic parameters were calculated using either Bioequiv (release 3.40) or WinNonLin™ (release 4.0.1). Bioequiv is proprietary software developed and tested for bioequivalence studies at SFBC Anapharm. This software performs non-compartmental analyses of pharmacokinetic parameters and statistical analyses (via SAS release 6.12) according to FDA, HPFB and BMEA guidance.

The mean, standard deviation (SD), coefficient of variation (CV (%)) and range (min. and max.) were calculated for plasma concentrations of P5P (both baseline corrected and uncorrected), PAL and PA for each sampling time and treatment.

For the non-compartmental pharmacokinetic analyses the mean. SD, CV (%). range (min. and max.), median and interquartile range were calculated for the AUC₀₋ₜ, (ng·h/mL), AUC₀₋ₜ (ng·h/mL), AUC_{0-inf} (ng·h/mL), Cₘₐₓ (ng/mL), Cₘᵢₙ (ng/mL), C_{avg} (ng/mL), Fl(%), Ae₀₋ₜ (ng), AUC^{t/inf} (%), Tₘₐₓ (h), T_{1/2 el} (h), Kₑₗ (h⁻¹), TLIN (h), LQCT (h). CL_{T} (L/h), CL_{R} (L/h). V_{β} (L), MRT (h) and Accumulation ratio. The calculation of these pharmacokinetic parameters is explained below.

### Maximum Observed Concentration, Minimum Observed Concentration, Average Concentration and Time of Observed Peak Concentration

Cₘₐₓ the maximum observed concentration, and Tₘₐₓ, the time to reach that peak concentration, were determined for each subject and for each analyte. For Day 4 data, Cₘᵢₙ the minimum observed concentration was determined for each subject and for each analyte and C_{avg} was calculated as AUC_{0-τ}/τ.

Percentage of Fluctuation—For Day 4 data, the peak-through percentage of fluctuation over one dosing interval (Fl(%)) was calculated as: 100*([Cₘₐₓ−Cₘᵢₙ]/C_{avg})

Half-Life and Elimination Rate Constant—To calculate the elimination rate constant (Kₑₗ), regression analyses were performed on the natural log(Ln) of plasma concentration values (y) versus time (x). Calculations were made between a time point where log-linear elimination phase begins (TLIN) and the time at which the last concentration above the limit of quantification (LQCT) occurred. The Kₑₗ was taken as the slope multiplied by (−1) and the apparent half-life (T_{1/2 el}) as (In 2)/Kₑₗ.

TLIN and LQCT—TLIN, the time point where In-linear Kₑₗ calculation begins, and LQCT, the sampling time of the last quantifiable concentration used to estimate the Kₑₗ were determined by the scientist (according to SFBC Anapharm's standard operating procedures) for each subject and for each treatment. At least 4 non-zero observations during the terminal elimination phase were used to calculate the Kₑₗ. A minimum of 3 observations were used if less than 4 observations were available.

Areas Under the Concentration-Time Curves—AUC₀₋ₜ was calculated using the linear trapezoidal rule from time 0.000 h until the last non-zero concentration.

AUC_{0-τ} was calculated using the linear trapezoidal rule from time 0.000 h until 24.0 h.

The AUC_{0-inf} was calculated as:

AUC₀₋ₜ+Cₜ/Kₑₗ

Where: Cₜ=the last observed non-zero concentration for that treatment, AUC₀₋ₜ=the AUC from time zero to the time of the last non-zero concentration for that analyte and Kₑₗ=the elimination rate constant.

AUC_{t/inf} was calculated as the ratio of AUCₒ₋ₜ (or AUC_{0-τ}) to AUC_{0-inf}.

Total and Renal Clearance—Total body clearance was estimated for P5P and was calculated as Dose/AUC_{0-inf}. Renal clearance was estimated for all analytes and was calculated as Ae₀₋ₜ/AUC_{0-inf} where Ae₀₋ₜ is the cumulative urinary excretion from 0.000 h until 24.0 h.

Volume of Distribution—Volume of distribution based on the terminal phase (Vβ) was calculated as Dose/(Kₑₗ*AUC_{0-inf}) for P5P and as Ae₀₋ₜ/(Kₑₗ*AUC_{0-inf}) for PAL and P A. For Day 4 data. AUC o−τ was used instead of AUC_{0-inf}.

Mean Residence Time—The mean residence time was calculated as AUMC/AUC_{0-inf}.

Accumulation Ratio—For Day 4 data, the theoretical accumulation ratio (using the studied dosing interval and assuming dose-linear kinetics) was calculated as 1/[1−e^{(-kei*τ)}].

Statistical Analysis—For all analytes, analysis of variance was performed on the ln transformed data of AUC₀₋ₜ, AUC_{0-inf} and Cₘₐₓ. All ANOVAs were performed with the SAS (release 6.12 for Windows) General Linear Models Procedure (GLM). The model included subject and treatment day. All sums of squares (Types I, II, III, and IV) were reported. The subject and day effects were tested against the residual mean square error. Probability (P) values were derived from Type III sums of squares. For all analyses, effects were considered statistically significant if the probability associated with “F” less than 0.050. Based on pairwise comparisons of the In-transformed of AUC₀₋ₜ, AUC_{0-inf} and Cₘₐₓ data, the ratios of the least squares means, calculated according to the formula “e^{(X-Y)}×100”, as well as the 90% geometric confidence intervals for In-transformed AUC₀₋ₜ, AUC_{0-inf} and Cₘₐₓ were determined. Finally, the intrasubject CVs were also determined.

### Analysis of Pharmacokinetics and Statistical Issues

### Non-Compartmental Pharmacokinetic Analysis

The primary non-compartmental pharmacokinetic parameters for this study were: AUC₀₋ₜ, AUC_{0-inf}, and Cₘₐₓ and the secondary pharmacokinetic parameters were AUC_{t/inf}, Tₘₐₓ, Kₑₗ, T_{1/2 el}, CL_{T} (for P5P), CL_{R}, V_{β}, MRT, and Ae₀₋ₜ. The following parameters were also calculated for Day 4 data: Cₘᵢₙ, C_{avg}, Fl(%), AUC_{0-τ}, and Accumulation ratio.

Blood sampling for pharmacokinetic analysis on Days 1 and 4 were collected at pre-dose and 0.083, 0.167. 0.250, 0.500, 0.750, 1.00, 1.50, 2.00, 3.00, 4.00, 6.00, 8.00, 12.0, and 24.0 hours post-dose on each day. Urine samples were collected at 8 time intervals: at 0.000-4.00, 4.00-8.00, 8.00-12.0, and 12.0-24.0 hours post-dose on Days 1 and 4.

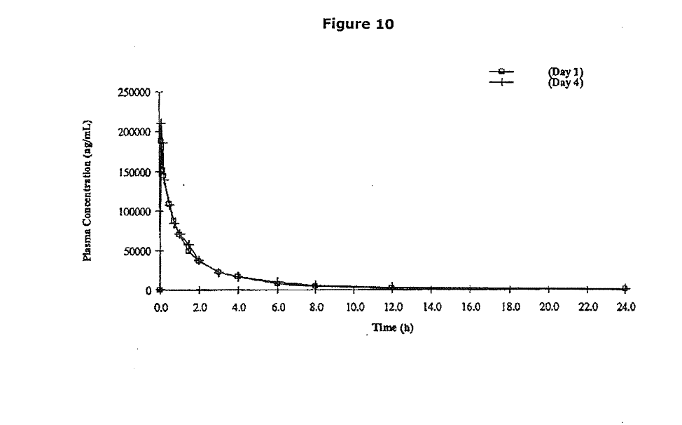

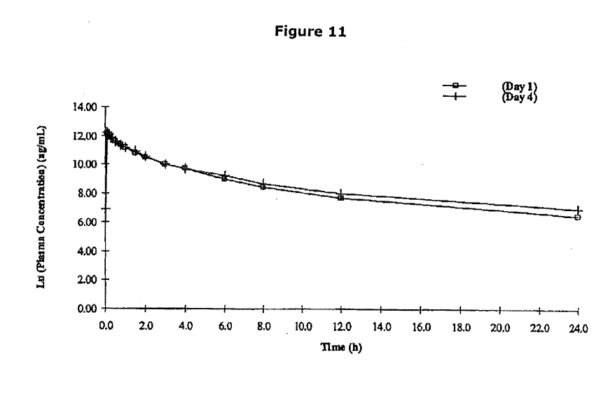

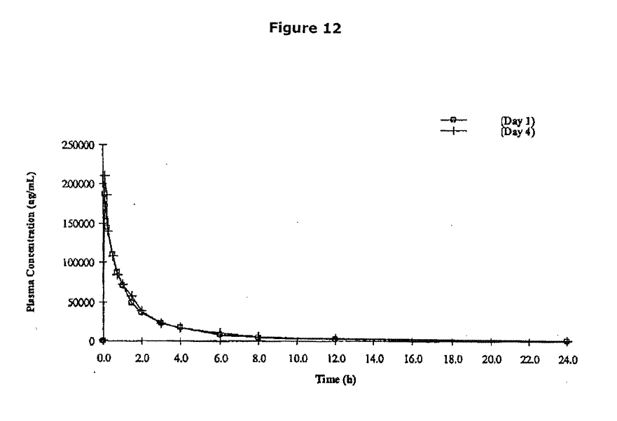

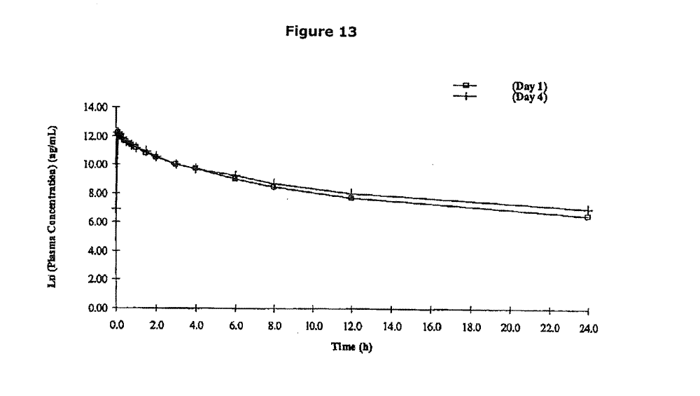

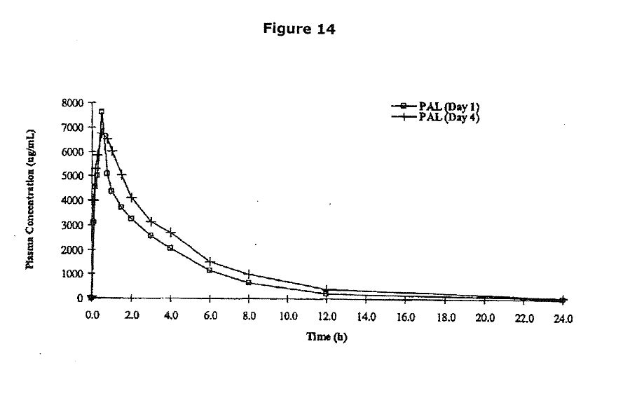

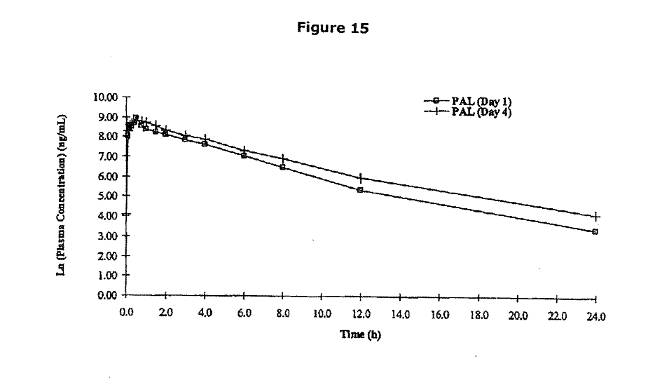

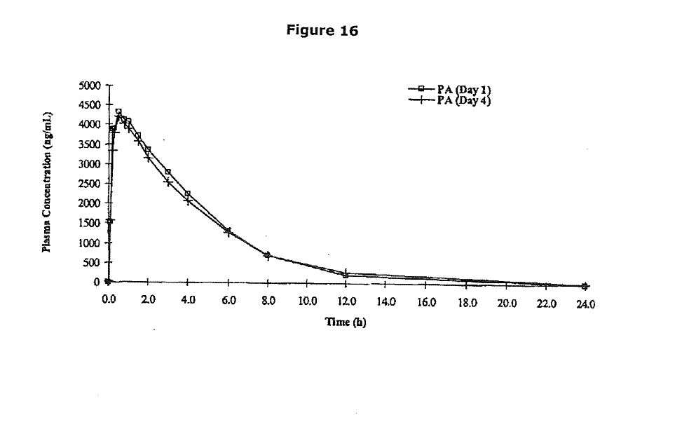

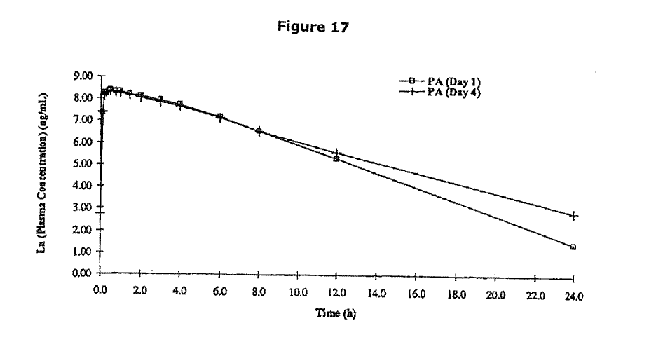

The plasma concentrations measured for each subject at each sampling time appear in Tables 9 and 10 for baseline corrected P5P, in Tables 11 and 12 for uncorrected P5P, in Tables 13 and 14 for PAL and in Tables 14 and 15 for PA according to treatment day. The plots of the mean plasma levels over the sampling period are presented for both untransformed and In-transformed in FIGS. 10-11 for baseline corrected P5P, in FIGS. 12-13 for uncorrected P5P, in FIGS. 14-15 for PAL and in FIGS. 16-17 for P A. The lines in In-transformed data figures represent the regression lines used to estimate the Kₑₗ.

TABLE 9
Summary of Pharmacokinetic Parameters for P5P (Corrected)

| | | P5P (Day 1) (A) | | | P5P (Day 4) (B) | | |
|---|---|---|---|---|---|---|---|
| Parameters | | Mean ± | SD | CV (%) | Mean ± | SD | CV (%) |
| AUC₀₋ₜ | (ng · h/mL) | 277978.56 ± | 54823.24 | 19.72 | 302742.00 ± | 85962.45 | 28.39 |
| AUC_{0-τ} | (ng · h/mL) | — | — | — | 302742.00 ± | 85962.45 | 28.39 |
| AUC_{0-inf} | (ng · h/mL) | 283108.04 ± | 58590.23 | 20.70 | 311164.84 ± | 90706.89 | 29.09 |
| AUC_{t/inf} | (%) | 98.33 ± | 0.95 | 0.96 | 97.23 ± | 1.02 | 1.05 |
| Cₘₐₓ | (ng/mL) | 190536 ± | 27999 | 14.69 | 213392 ± | 46782 | 21.92 |
| Cₘᵢₙ | (ng/mL) | — | — | — | 946 ± | 582 | 61.50 |
| C_{avg} | (ng/mL) | — | — | — | 12614.42 ± | 3582.24 | 28.40 |
| Fl (%) | (%) | — | — | — | 1734.69 ± | 378.44 | 21.82 |
| Tₘₐₓ | (h) | 0.089 ±: | 0.040 | 45.25 | 0.081 ± | 0.034 | 41.92 |
| Tₘₐₓ | (h) | 0.067 ± | 0.025 | — | 0.067 ± | 0.000′ | — |
| Kₑₗ | (h⁻¹) | 0.1381 ± | 0.0226 | 16.38 | 0.1183 ± | 0.0170 | 14.41 |
| T_{1/2 el} | (h) | 5.14 ± | 0.91 | 17.65 | 5.96 ± | 0.85 | 14.33 |
| CL_{τ} | (L/h) | 2.55 ± | 0.38 | 14.75 | 2.42 ± | 0.38 | 15.82 |
| V_{β} | (L) | 18.59 ± | 1.87 | 10.05 | 20.75 ± | 4.08 | 19.65 |
| MRT | (h) | 3.40 ± | 0.70 | 20.68 | 3.99 ± | 0.67 | 16.84 |

| | | | | | | |
|---|---|---|---|---|---|---|
| Accumulation Ratio | — | — | — | 1.07 ± | 0.03 | 2.62 |

TABLE 10
Summary of Non-Compartmental Pharmacokinetic
Parameters for P5P (Corrected)
P5P (Day 4) (B) vs P5P (Day 1) (A)

| | AUC₀₋ₜ | AUC_{0-inf} | Cₘₐₓ |
|---|---|---|---|
| Ratio¹ | 107.31% | 108.53% | 110.71% |
| 90% Geometric | 98.28% to 117.16% | 99.88%10117.91% | 98.76% to |
| C.I.² | | | 124.10% |
| Intra-Subject CV | 7.56% | 7.14% | 9.84% |

¹Calculated using least-squares means according to the formula: e^{(P5P (Day 4) (B)-P5P (Day 1) (A))} × 100
²90% Geometric Confidence Interval using ln-transformed data

TABLE 11
Summary of Pharmacokinetic Parameters for P5P
(Uncorrected)

| | | P5P (Day 1) (A) | | | P5P (Day 4) (B) | | |
|---|---|---|---|---|---|---|---|
| Parameters | | Mean ± | SD | CV (%) | Mean ± | SD | CV (%) |
| AUC₀₋ₜ | (ng · h/mL) | 278624.79 ± | 55320.00 | 19.85 | 303392.72 ± | 86509.09 | 28.51 |
| AUC_{0-τ} | (ng · h/mL) | — | — | — | 303393.72 ± | 86509.09 | 28.51 |
| AUC_{0-inf} | (ng · h/mL) | 284071.57 ± | 59408.49 | 20.91 | 312753.76 ± | 91554.52 | 29.27 |
| AUC_{t/inf} | (%) | 98.24 ± | 1.01 | 1.03 | 97.14 ± | 1.04 | 1.07 |
| Cₘₐₓ | (ng/mL) | 190564 ± | 27998 | 14.69 | 213419 ± | 46789 | 21.92 |
| Cₘᵢₙ | (ng/mL) | — | — | — | 974 ± | 603 | 61.89 |
| C_{avg} | (ng/mL) | — | — | — | 12641.59 ± | 3605.03 | 28.52 |
| Fl (%) | (%) | 848625 ± | 612120 | 72.13 | 1731.62 ± | 378.85 | 21.88 |
| Tₘₐₓ | (h) | 0.089 ± | 0.040 | 45.25 | 558650 ± | 414503 | 74.20 |
| Tₘₐₓ | (h) | 0.067 ± | 0.025 | — | 0.081 ± | 0.034 | 41.92 |
| Kₑₗ | (h⁻¹) | 0.1360 ± | 0.0226 | 16.60 | 0.067 ± | 0.000 | — |
| T_{1/2 el} | (h) | 5.22 ± | 0.94 | 18.00 | 0.1170 ± | 0.0168 | 14.35 |
| CL_{τ} | (L/h) | 2.55 ± | 0.38 | 14.92 | 6.02 ± | 0.85 | 14.14 |
| V_{β} | (L) | 0.00279 ± | 0.00177 | 63.53 | 2.42 ± | 0.38 | 15.92 |
| MRT | (h) | 18.82 ± | 1.89 | 10.03 | 0.00174 ± | 0.0078 | 44.71 |

| | | | | | | |
|---|---|---|---|---|---|---|
| Accumulation Ratio | 3.45 ± | 0.73 | 21.20 | 20.92 ± | 4.05 | 19.36 |
| | — | — | — | 4.03 ± | 0.68 | 16.93 |
| | | | | 1.07 ± | 0.03 | 2.63 |

TABLE 12
Summary of Non-Compartmental Pharmacokinetic
Parameters for P5P (Uncorrected)
P5P (Day 4) (B) vs P5P (Day 1) (A)

| | AUC₀₋ₜ | AUC_{0-inf} | Cₘₐₓ |
|---|---|---|---|
| Ratio¹ | 107.28% | 108.50% | 110.70% |
| 90% Geometric | 98.28% to 117.11% | 99.90% to 117.83% | 98.76% to |
| C.I.² | | | 124.10% |
| Intra-Subject CV | 7.54% | 7.10% | 9.84% |

¹Calculated using least-squares means according to the formula: e^{(P5P (Day 4) (B)-P5P (Day 1) (A))} × 100
²90% Geometric Confidence Interval using ln-transformed data

TABLE 13
Summary of Pharmacokinetic Parameters for PAL

| | | PAL (Day 1) (A) | | | PAL (Day 4) (B) | | |
|---|---|---|---|---|---|---|---|
| Parameters | | Mean ± | SD | CV (%) | Mean ± | SD | CV (%) |
| AUC₀₋ₜ | (ng · h/mL) | 22292.93 ± | 3412.91 | 15.31 | 29682.94 ± | 5021.44 | 16.92 |
| AUC_{0-τ} | (ng · h/mL) | — | — | — | 29682.94 ± | 5021.44 | 16.92 |
| AUC_{0-inf} | (ng · h/mL) | 22444.06 ± | 3396.70 | 15.13 | 30077.45 ± | 5010.46 | 16.66 |
| AUC_{t/inf} | (%) | 99.31 ± | 0.41 | 0.41 | 98.66 ± | 0.87 | 0.89 |
| Cₘₐₓ | (ng/mL) | 7629 ± | 6075 | 79.63 | 6902 ± | 2936 | 42.54 |
| Cₘᵢₙ | (ng/mL) | — | — | — | 56.8 ± | 28.3 | 49.95 |
| C_{avg} | (ng/mL) | — | — | — | 1236.81 ± | 209.24 | 16.92 |
| Fl (%) | (%) | — | — | — | 537.81 ± | 151.79 | 28.22 |
| Tₘₐₓ | (h) | 18583712 ± | 6900473 | 37.13 | 21011635 ± | 4491980 | 21.38 |
| Tₘₐₓ | (h) | 0.525 ± | 0.102 | 19.45 | 0.691 ± | 0.188 | 27.22 |
| Kₑₗ | (h⁻¹) | 0.483 ± | 0.000 | — | 0.733 ± | 0.188 | — |
| T_{1/2 el} | (h) | 0.2064 ± | 0.0262 | 12.70 | 0.1775 ± | 0.0236 | 13.27 |
| CL_{τ} | (L/h) | 3.40 ± | 0.41 | 12.11 | 3.96 ± | 0.54 | 13.56 |
| V_{β} | (L) | 0.85952 ± | 0.37133 | 43.20 | 0.72392 ± | 0.19711 | 27.23 |
| MRT | (h) | 4.35 ± | 2.18 | 50.05 | 4.21 ± | 1.63 | 38.82 |

| | | | | | | |
|---|---|---|---|---|---|---|
| Accumulation Ratio | 4.06 ± | 0.70 | 17.24 | 4.74 ± | 0.96 | 20.29 |
| | — | — | — | 1.02 ± | 0.01 | 0.91 |

TABLE 14
Summary of Non-Compartmental Pharmacokinetic
Parameters for PAL
PAL (Day 4) (B) vs PAL (Day 1) (A)

| | AUC₀₋ₜ | AUC_{0-inf} | |
|---|---|---|---|
| Ratio¹ | 132.83% | 133.70% | 101.45% |
| 90% | 128.10% to 137.73% | 128.85% to 138.74% | 79.57% to |
| Geometric | | | 129.33% |
| C.I.² | | | |
| Intra-Subject | 3.12% | 3.18% | 21.10% |
| CV | | | |

¹Calculated using least-squares means according to the formula: e^{(PAL (Day 4) (B)-PAL (Day 1) (A))} × 100
²90% Geometric Confidence Interval using ln-transformed data

TABLE 15
Summary of Pharmacokinetic Parameters for PA

| | | PA (Day 1) (A) | | | PA (Day 4) (B) | | |
|---|---|---|---|---|---|---|---|
| Parameters | | Mean ± | SD | CV (%) | Mean ± | SD | CV (%) |
| AUC₀₋ₜ | (ng · h/mL) | 20736.28 ± | 3586.19 | 17.29 | 20704.80 ± | 3714.71 | 17.94 |
| AUC_{0-τ} | (ng · h/mL) | — | — | — | 21240.85 ± | 3451.14 | 16.25 |
| AUC_{0-inf} | (ng · h/mL) | 21219.58 ± | 3686.62 | 17.37 | 21097.95 ± | 3614.47 | 17.13 |
| AUC_{t/inf} | (%) | 97.73 ± | 1.21 | 1.24 | 98.01 ± | 1.42 | 1.45 |
| Cₘₐₓ | (ng/mL) | 4362 ± | 797 | 18.27 | 4238 ± | 771 | 18.20 |
| Cₘᵢₙ | (ng/mL) | — | — | — | 12.9 ± | 25.1 | 194.30 |
| C_{avg} | (ng/mL) | — | — | — | 885.04 ± | 143.80 | 16.25 |
| Fl (%) | (%) | — | — | — | 482.53 ± | 82.29 | 17.05 |
| Tₘₐₓ | (h) | 172950596 ± | 66502786 | 38.45 | 225885311 ± | 47312304 | 20.95 |
| Tₘₐₓ | (h) | 0.611 ± | 0.215 | 35.24 | 0.528 ± | 0.101 | 19.13 |
| Kₑₗ | (h⁻¹) | 0.483 ± | 0.188 | — | 0.483 ± | 0.013 | — |
| T_{1/2 el} | (h) | 0.3092 ± | 0.0526 | 17.01 | 0.2547 ± | 0.0524 | 20.59 |
| CL_{τ} | (L/h) | 2.30 ± | 0.45 | 19.38 | 2.83 ± | 0.67 | 23.55 |
| V_{β} | (L) | 8.61246 ± | 4.23700 | 49.20 | 10.75339 ± | 2.18960 | 20.36 |
| MRT | (h) | 28.61 ± | 14.16 | 49.49 | 44.45 ± | 14.95 | 33.63 |

| | | | | | | |
|---|---|---|---|---|---|---|
| Accumulation Ratio | 3.85 ± | 0.61 | 15.94 | 4.24 ± | 0.92 | 21.69 |
| | — | — | — | 1.00 ± | 0.01 | 0.58 |

TABLE 16
Summary of Non-Compartmental Pharmacokinetic
Parameters for PA
PA (Day 4) (B) vs PA (Day 1) (A)

| | AUC₀₋ₜ | AUC_{0-inf} | Cₘₐₓ |
|---|---|---|---|
| Ratio¹ | 99.75% | 99.47% | 97.17% |
| 90% Geometric | 95.89% to 103.76% | 94.73% to 104.46% | 94.16% to |
| C.I.² | | | 100.28% |
| Intra-Subject CV | 3.39% | 4.20% | 2.71% |

¹Calculated using least-squares means according to the formula: e^{(PA (Day 4) (B)-PA (Day 1) (A))} × 100
²90% Geometric Confidence Interval using ln-transformed data

Calculated non-compartmental pharmacokinetic parameters for each subject according to treatment day are shown in Tables 10, 12, 14, and 16 for corrected and uncorrected P5P, PAL, and PA.

### Primary Parameters

Area under the concentration-time curve (O-t hours) was calculated for each subject and analyte. The P5P (baseline corrected) results are presented in Tables 9 and 10. Mean values (% CV) for AUC₀₋ₜ were 277978.56 ng·h/mL (19.72%) for Day 1 and 302742.00 ng·h/mL (28.39%) for Day 4. The P5P (uncorrected) results are presented in Tables 11 and 12. Mean values (% CV) for AUC₀₋ₜ were 278624.79 ng·h/mL (19.85%) for Day 1 and 303393.72 ng·h/mL (28.51%) for Day 4. The PAL results are presented in Tables 11 and 12. Mean values (% CV) for AUC₀₋ₜ were 22292.93 ng·h/mL (15.31%) for Day 1 and 29682.94 ng·h/mL (16.92%) for Day 4. The PA results are presented in Tables 11 and 12. Mean values (% CV) for AUC₀₋ₜ were 20736.28 ng·h/mL (17.29%) for Day 1 and 20704.80 ng·h/mL (17.94%) for Day 4. The ANOVA performed on the In-transformed AUC₀₋ₜ data are presented in Tables 710, 12, 14, and 16 for P5P (baseline corrected), P5P (uncorrected), PAL and PA, respectively. ANOVA did not detect any statistically significant difference between Day 4 and Day 1 for this parameter for P5P (baseline corrected and uncorrected) and PA. ANOVA detected a statistically significant difference between Day 4 and Day 1 for this parameter for PAL. The least-squares means ratios, the 90% geometric confidence intervals and intra-subject CVs were also determined for P5P (baseline corrected), P5P (uncorrected), PAL and P A, respectively. These results are summarized on Table 17.

TABLE 17
Summary of least-squares means ratios, 90% geometric
confidence intervals and intra-subject CVs for AUC₀₋ₜ

| | Treatment Day 4 (B) vs Treatment Day 1 (A) | | | |
|---|---|---|---|---|
| | P5P | | | |
| | Baseline | P5P | | |
| AUC₀₋ₜ | corrected | Uncorrected | PAL | PA |
| Ratio of LS Means | 107.31% | 107.28% | 132.83% | 99.75% |
| 90% Geometric C.I. | 98.28% to | 98.28% to | 12&.IO % to | 95.89% to |
| | 117.16% | 117.11% | 137.73% | 103.76% |
| Inter-Subject CV | 7.56% | 754% | 3.12% | 3.39% |

Area under the concentration-time curve (0-infinity) was calculated for each subject and analyte. The P5P (baseline corrected) results are presented in Table 9. Mean values (% CV) for AUC_{0-inf} were 283108.04 ng·h/mL (20.70%) for Day 1 and 311764.84 ng·h/mL (29.09%) for Day 4. The P5P (uncorrected) results are presented in Table 11. Mean values (% CV) for AUC_{0-inf} were 284071.57 ng·h/mL (20.91%) for Day 1 and 312753.76 ng·h/mL (29.27%) for Day 4. The PAL results are presented in Table 13. Mean values (% CV) for AUC_{0-inf} were 22444.06 ng·h/mL (15.13%) for Day 1 and 30077.45 ng·h/mL (16.66%) for Day 4. The PA results are presented in Table 15. Mean values (% CV) for AUC_{0-inf} were 21219.58 ng·h/mL (17.37%) for Day 1 and 21097.95 ng·h/mL (17.13%) for Day 4.

ANOVA did not detect any statistically significant difference between Day 4 and Day 1 for this parameter for P5P (baseline corrected and uncorrected) and PA. ANOVA detected a statistically significant difference between Day 4 and Day 1 for this parameter for PAL. The least-squares means ratios, the 90% geometric confidence intervals and intra-subject CVs were also determined for P5P (baseline corrected), P5P (uncorrected), PAL and PA, respectively. These results are summarized on Table 18.

TABLE 18
Summary of least-squares means ratios, 90% geometric
confidence intervals and intra-subject CVs for AUC_{0-inf}

| | Treatment Day 4 (B) vs Treatment Day 1 (A) | | | |
|---|---|---|---|---|
| | P5P | | | |
| | Baseline | P5P | | |
| AUC_{0-inf} | corrected | Uncorrected | PAL | PA |
| Ratio of LS Means | 108.53% | 108.50% | 133.70% | 99.47% |
| 90% Geometric C.I. | 99.88% to | 99.90% to | 128.85% to | 94.73% to |
| | 117.91% | 117.83% | 138.74% | 104.46% |
| Intra-Subject CV | 7.14% | 7.10% | 3.18% | 4.20% |

The peak or maximal plasma concentration was calculated for each subject and analyte. The P5P (baseline corrected) results are presented in Table 9. Mean values (% CV) for Cₘₐₓ were 190536 ng/mL (14.69%) for Day 1 and 213392 ng/mL (21.92%) for Day 4. The P5P (uncorrected) results are presented in Table 11. Mean values (% CV) for Cₘₐₓ were 190564 ng/mL (14.69%) for Day 1 and 213419 ng/mL (21.92%) for Day 4. The PAL results are presented in Table 13. Mean values (% CV) for Cₘₐₓ were 7629 ng/mL (79.63%) for Day 1 and 6902 ng/mL (42.54%) for Day 4. The PA results are presented in Table 15. Mean values (% CV) for Cₘₐₓ were 4362 ng/mL (18.27%) for Day 1 and 4238 ng/mL (18.20%) for Day 4. ANOVA did not detect any statistically significant difference between treatments for this parameter for all analytes. The least-squares means ratios, the 90% geometric confidence intervals and intra-subject CVs were also determined for P5P (baseline corrected), P5P (uncorrected), PAL and PA, respectively. These results are summarized on Table 19.

TABLE 19
Summary of least-squares means ratios, 90% geometric
confidence intervals and intra-subject CVs for Cₘₐₓ

| | Treatment Day 4 (B) vs Treatment Day 1 (A) | | | |
|---|---|---|---|---|
| | P5P | | | |
| | Baseline | P5P | | |
| Cₘₐₓ | corrected | Uncorrected | PAL | PA |
| Ratio of LS Means | 110.71% | 110.70% | 101.45% | 97.17% |
| 90% Geometric C.I. | 98.76% to | 98.76% to | 79.57% to | 94.16% to |
| | 124.10% | 124.10% | 129.33% | 100.28% |
| Intra-Subject CV | 9.84% | 9.84% | 21.10% | 2.71% |

### Secondary Parameters Common to Day 1 and Day 4

The ratio of AUC₀₋ₜ to AUC_{0-inf} (%) was calculated for each subject and for each analyte. The P5P (baseline corrected) results are presented in Table 9. Mean values (% CV) for AUC_{t/inf} were 98.33% (0.96%) for Day 1 and 97.23% (1.05%) for Day 4. The P5P (uncorrected) results are presented in Table 11. Mean values (% CV) for AUC_{t/inf} were 98.24% (1.03%) for Day 1 and 97.14% (1.07%) for Day 4. The PAL results are presented in Table 13. Mean values (% CV) for AUC_{t/inf} were 99.31% (0.41%) for Day 1 and 98.66% (0.89%) for Day 4. The PA results are presented in Table 15. Mean values (% CV) for AUC_{t/inf} were 97.73% (1.24%) for Day 1 and 98.01% (1.45%) for Day 4. The mean AUC_{t/inf} greater than 80% for all analytes, both on Day 1 and Day 4 indicated that the duration of sampling was sufficient.

The total amount excreted in urine (0-t hours) was calculated for each subject and analyte (except for P5P baseline corrected data). The P5P (uncorrected) results are presented in Table 11. Mean values (% CV) for Ae₀₋ₜwere 848625 ng (72.13%) for Day 1 and 558650 ng (74.20%) for Day 4. The PAL results are presented in Table 13. Mean values (% CV) for Ae₀₋ₜ were 18583712 ng (37.13%) for Day 1 and 21011635 ng (21.38%) for Day 4. The PA results are presented in Table 15. Mean values (% CV) for Ae₀₋ₜ were 172950596 ng (38.45%) for Day 1 and 225885311 ng (20.95%) for Day 4.

The time to reach the peak concentration was determined for each subject and analyte. The P5P (baseline corrected and uncorrected) results are summarized in Tables 6 and 8, respectively. Mean values (% CV) for the Tₘₐₓ were 0.089 h (45.25%) for Day 1 and 0.081 h (41.92%) for Day 4. The PAL results are summarized in Table 13. Mean values (% CV) for the Tₘₐₓ were 0.525 h (19.45%) for Day 1 and 0.691 h (27.22%) for Day 4. The PA results are summarized in Table 15. Mean values (% CV) for the Tₘₐₓ were 0.611 h (35.24%) for Day 1 and 0.528 h (19.13%) for Day 4.

The elimination rate constant was calculated for each subject and analyte. The P5P (baseline corrected) results are presented in Table 9. Mean values (% CV) for the Kₑₗ were 0.1381 h⁻¹ (16.38%) for Day 1 and 0.1183 h⁻¹ (14.41%) for Day 4. The P5P (uncorrected) results are presented in Table 11 and. Mean values (% CV) for the Kₑₗ were 0.1360 h⁻¹ (16.60%) for Day 1 and 0.1170 h⁻¹ (14.35%) for Day 4. The PAL results are presented in Table 13. Mean values. (% CV) for the Kₑₗ were 0.2064 h⁻¹ (12.70%) for Day 1 and 0.1775 h⁻¹ (13.27%) for Day 4. The PA results are presented in Table 15. Mean values (% CV) for the Kₑₗ were 0.3092 h⁻¹ (17.01%) for Day 1 and 0.2547 h⁻¹ (20.59%) for Day 4.

The apparent half-life was calculated for each subject and treatment day. The P5P (baseline corrected) results are presented in Table 9. Mean values (% Cv) for the T_{1/2 el} were 5.14 h (17.65%) for Day 1 and 5.96 h (14.33%) for Day 4. The P5P (uncorrected) results are presented in Table 11. Mean values (% CV) for the T_{1/2 el} were 5.22 h (18.00%) for Day 1 and 6.02 h (14.14%) for Day 4. The PAL results are presented in Table 13. Mean values (% CV) for the T_{1/2 el} were 3.40 h (12.11%) for Day 1 and 3.96 h (13.56%) for Day 4. The PA results are presented in Table 15. Mean values (% CV) for the T_{1/2 el} were 2.30 h (19.380%) for Day 1 and 2.83 h (23.55%) for Day 4.

Total body clearance was calculated for each subject for P5P (baseline corrected and uncorrected), The P5P (baseline corrected) results are presented in Table 9. Mean values (% CV) for the CL_{T} were 2.55 L/h (14.75%) for Day 1 and 2.42 L/h (15.82%) for Day 4, The P5P (uncorrected) results are presented in Table 11. Mean values (% CV) for the CL_{T} were 2.55 L/h (14.92%) for Day 1 and 2.42 L/h (15.92%) for Day 4.

Renal clearance was calculated for each subject and analyte (except baseline corrected P5P). The P5P (uncorrected) results are presented in Table 11. Mean values (% CV) for the CL_{R} were 0.00279 L/h (63.53%) for Day 1 and 0.00174 L/h (44.71%) for Day 4. The PAL results are presented in Table 13. Mean values (% CV) for the CLR were 0.85952 L/h (43.20%) for Day 1 and 0.72392 L/h (27.23%) for Day 4. The PA results are presented in Table 15. Mean values (% CV) for the CL_{R} were 8.61246 L/h (49.20%) for Day 1 and 10.75339 L/h (20.36%) for Day 4.

Volume of distribution based on the terminal phase was calculated for each subject and analyte. The P5P (baseline corrected) results are presented in Tables 6. Mean values (% CV) for the V_{β} were 18.59 L (10.05%) for Day 1 and 20.75 L (19.65%) for Day 4. The P5P (uncorrected) results are presented in Table 11. Mean values (% CV) for the V_{β} were 18.82 L (10.03%) for Day 1 and 20.92 L (19.36%) for Day 4. The PAL results are presented in Tables 10. Mean values (% CV) for the V_{β} were 4.35 L (50.05%) for Day 1 and 4.21 L (38.82%) for Day 4. The PA results are presented in Table 15. Mean values (% CV) for the V_{β} were 28.61 L (49.49%) for Day 1 and 44.45 L (33.63%) for Day 4.

The mean residence time was calculated for each subject and analyte, The P5P (baseline corrected) results are presented in Table 9. Mean values (% CV) for the MRT were 3.40 h (20.68%) for Day 1 and 3.99 h (16.84%) for Day 4. The P5P (uncorrected) results are presented in Table 11. Mean values (% CV) for the MRT were 3.45 h (21.20%) for Day 1 and 4.03 h (16.93%) for Day 4. The PAL results are presented in Table 13. Mean values (% CV) for the MRT were 4.06 h (17.24%) for Day 1 and 4.74 h (20.29%) for Day 4. The PA results are presented in Table 15. Mean values (% CV) for the MRT were 3.85 h (15.94%) for Day 1 and 4.24 h (21.69%) for Day 4.

### Secondary Parameters Specific to Day 4

The minimum or through concentration was obtained for each subject and analyte on Day 4. The P5P (baseline corrected) results are presented in Table 3. Mean value (% CV) for the Cₘᵢₙ was 946 ng/mL (61.50%). The P5P (uncorrected) results are presented in Table 11. Mean value (% CV) for the Cₘᵢₙ was 974 ng/mL (61.89%). The PAL results are presented in Table 13. Mean value (% CV) for the Cₘᵢₙ was 56.8 ng/mL (49.95%). The PA results are presented in Table 15. Mean value (% CV) for the Cₘᵢₙ was 12.9 ng/mL (194.30%).

The average concentration was obtained for each subject and analyte on Day 4. The P5P (baseline corrected) results are presented in Table 9. Mean value (% CV) for the C_{avg} was 12614.42 ng/mL (28.40%). The P5P (uncorrected) results are presented in Table 11. Mean value (% CV) for the C_{avg} was 12641.59 ng/mL (28.52%). The PAL results are presented in Table 13. Mean value (% CV) for the C_{avg} was 1236.81 ng/mL (16.92%). The PA results are presented in Table 15. Mean value (% CV) for the C_{avg} was 885.04 ng/mL (16.25%).

The percentage of fluctuation was obtained for each subject and analyte on Day 4. The P5P (baseline corrected) results are presented in Table 9. Mean value (% CV) for the Fl(%) was 1734.69% (21.82%). The P5P (uncorrected) results are presented in Table 11. Mean value (% CV) for the Fl(%) was 1731.62% (21.88%). The PAL results are presented in Table 13. Mean value (% CV) for the Fl(%) was 537.81% (28.22%). The PA results are presented in Table 15. Mean value (% CV) for the Fl(%) was 482.53% (17.05%).

Area under the concentration-time curve (0-tau) was calculated for each subject and analyte on Day 4. The P5P (baseline corrected) results are presented in Table 9. Mean value (% CV) for the AUC_{0-τ} was 302742.00 ng·h/mL (28.39%). The P5P (uncorrected) results are presented in Table 11. Mean value (% CV) for the AUC_{0-τ} was 303393.72 ng·h/mL (28.51%). The PAL results are presented in Table 13. Mean value (% CV) for the AUC_{0-τ} was 29682.94 ng·h/mL (16.92%). The PA results are presented in Table 15. Mean value (% CV) for the AUC_{0-τ} was 21240.85 ng·h/mL (16.25%).

Theoretical accumulation ratio was calculated for each subject and analyte on Day 4. The P5P (baseline corrected) results are presented in Table 9. Mean value (% CV) for the accumulation ratio was 1.07 (2.62%). The P5P (uncorrected) results are presented in Table 11. Mean value (% CV) for the accumulation ratio was 1.07 (2.63%). The PAL results are presented in Table 13. Mean value (% CV) for the accumulation ratio was 1.02 (0.91%). The PA results are presented in Table 15. Mean value (% CV) for the accumulation ratio was 1.00 (0.58%).

### Non-Compartmental Pharmacokinetic and Statistical Conclusions

ANOVA did not detect any statistically significant difference between treatment days for In-transformed AUC₀₋ₜ, AUC_{0-inf} and Cₘₐₓ for P5P (both baseline corrected and uncorrected) and PA.

For PAL, a statistically significant difference between treatment days was detected using ANOVA for In-transformed AUC₀₋ₜ, and AUC_{0-inf} but not for Cₘₐₓ.

The mean AUC_{t/inf} greater than 80% for all analytes indicated that the duration of sampling was sufficient to well characterize their kinetics. The intra-subject CVs for AUC₀₋ₜ, AUC_{0-inf} and Cₘₐₓ were respectively 7.56%, 7.14% and 9.84% for P5P (baseline corrected), 7.54%, 7.10% and 9.84% for P5P (uncorrected), 3.12%, 3.18% and 21.10% for PAL and 3.390%, 4.20% and 2.71% for PA.

Results of the 90% geometric confidence intervals of the ratio of least-squares means of the test to reference product of In-transformed AUC₀₋ₜ, AUC_{0-inf} and Cₘₐₓ were within the range of 80.00% to 125.00% for P5P (both baseline corrected and uncorrected) and PA but not for PAL. For P5P and PA, these results suggest that with a once daily dosing regimen, accumulation is not likely to occur and changes in rate and extent of absorption (for P5P) or biotransformation (for PA) should not be significant. For PAL, while the theoretical accumulation ratio is close to 1, it becomes apparent looking at the individual concentration-time profiles that accumulation is occurring following four days of once daily dosing of P5P.

### Safety Evaluation

Six (6) subjects experienced a total of 40 adverse events during this: study. Inflammation at injection site was reported by 3 subjects (50.0% of subjects). These adverse events, however, were judged by a Medical Sub-Investigator, to be associated to the procedures and were recorded as unrelated to the study medication. Of the remaining, potentially associated, adverse events reported during the study the following adverse events were reported by more than one subject: nausea, dizziness (feels dizzy and dizziness), paresthesia (feels like pins and needles on tip of first finger [right hand] and tingling in back of neck, and feels cold [right hand]) and white blood cells in urine. The remaining adverse events were reported by only one subject: low hemoglobin, feels anxious, bruise at catheter site (right hand), red blood cells in urine, muscular cramps on thighs, low neutrophils in blood, pain (both legs), lower back pain, pain in back of neck, papules on both sides of the chest, more saliva in mouth, sweating of body, fainting, abnormal taste in the mouth, and feels hot.

Nausea, dizziness (feels dizzy and dizziness), and abnormal taste in the mouth were expected adverse events. Dizziness (feels dizzy and dizziness) was reported by 2 subjects (33.3% of subjects). Nausea was reported by 3 subjects (50.0% of subjects). Abnormal taste in the mouth was reported by 1 subject (16.7%); the maximal severity was mild for three subjects (Subjects No. 01 to 04) and moderate (Subject No. 04) for one subject. No diarrhea or vomiting was reported during this study.

Clinically significant laboratory results for elevated white blood cells (WBC) in urine (pyuria) were observed for two subjects. One subject (#03) had negative results for leukocyte esterase at the time of screening and Days—1 to 3 (because leukocyte esterase was negative, no microscopic evaluation was performed at that time). On Day 4, this subject presented with + for leukocyte esterase and 3-5/HPF for WBC; on Day 5, leukocyte esterase remained at +, but WBC rose to 10/20/HPF. A follow-up test, performed approximately 11 days later yielded a normal result. The other subject (#06) presented with trace results for leukocyte esterase and 3-5/HPF for WBC at the time of screening, +for leukocyte esterase and 0-2/HPF for WBC on Day 1, and + for leukocyte esterase and negative for WBC on Day 2; however, results for leukocyte esterase were negative on Days—1 and 3. On Day 4, this subject presented with ++ for leukocyte esterase and 10-20/HPF for WBC; on Day 5 leukocyte esterase had diminished to + and WBC to 5-10/HPF.

### CONCLUSIONS

No serious, severe, or significant adverse events were reported during this study_Upon conclusion of the clinical portion of the study, the results from the post-study laboratory tests, physical examinations, vital signs measurements, and ECGs confirmed the absence of significant changes in the subjects' state of health. Overall, the safety profile of this formulation established in this clinical trial demonstrates that the product is well tolerated with no serious or lasting treatment related effects.

Results of the 90% geometric confidence intervals of the ratio of least squares means of the test to reference product of In-transformed AUC₀₋ₜ>AUC_{0-inf} and Cₘₐₓ were within the range of 80.00% to 125.00% for P5P (both baseline corrected and uncorrected) and PA but not for PAL. For P5P and PA, these results suggest that with a once daily dosing regimen, accumulation is not likely to occur and changes in rate and extent of absorption (for P5P) or biotransformation (for PA) should not be significant. For PAL, while the theoretical accumulation ratio is close to 1, it becomes apparent looking at the individual concentration-time profiles that accumulation is occurring following four days of once daily dosing of P5P.

1. A lyophilized formulation of pyridoxal 5′-phosphate prepared by lyophilizing a frozen sterile aqueous solution of pyridoxal 5′-phosphate in a concentration higher than a supplement concentration and sodium hydroxide.

2. The lyophilized formulation according to claim 1, wherein the pH of the solution is between 7.0 and 7.3.

3. The lyophilized formulation according to claim 1, further comprising mannitol.

4. The lyophilized formulation according to claim 1, wherein the sterile solution contains about 1 to 25% w/w pyridoxal 5′-phosphate.

5-7. (canceled)

8. The lyophilized formulation according to claim 1, wherein the sterile solution contains about 0.1 to 10% w/w sodium hydroxide.

9-11. (canceled)

12. The lyophilized formulation according to claim 1, wherein the sterile solution contains about 0.1 to 15% w/w mannitol.

13-15. (canceled)

16. The lyophilized formulation according to claim 1, wherein the sterile solution contains about 5% w/w pyridoxal 5′-phosphate, about 1.5% w/w of the sodium hydroxide and about 2.8% w/w of the mannitol.

17. The lyophilized formulation according to claim 1, wherein the sterile solution contains about 250 mg pyridoxal 5′-phosphate, about 80.5 mg sodium hydroxide and about 150 mg of mannitol.

18. An injectable formulation comprising pyridoxal 5′-phosphate, reconstituted from a lyophilized formulation according to claim 1, using a sterile carrier suitable for intravenous administration.

19. The injectable formulation according to claim 18, wherein the sterile carrier is water for injection.

20. A process for preparing a lyophilized formulation of pyridoxal 5′-phosphate comprising:
(a) preparing a sterile solution comprising pyridoxal 5′-phosphate and sodium hydroxide, said solution having a pH about 7.0 to 7.3; and
(b) freezing the solution; and
(c) lyophilizing the frozen solution.

21. The process according to claim 20, wherein step (a) further comprises dissolving mannitol in the sterile solution of pyridoxal 5′-phosphate and sodium hydroxide.

22. The process according to claim 20, wherein the sterile solution contains pyridoxal 5′-phosphate in a concentration higher than a supplement concentration.

23. The process according to claim 20, wherein the sterile solution contains about 1 to 25% w/w pyridoxal 5′-phosphate.

24-26. (canceled)

27. The process according to claim 20, wherein the sterile solution contains about 0.1 to 10% w/w sodium hydroxide.

28-30. (canceled)

31. The process according to claim 20, wherein the sterile solution contains about 0.1 to 15% w/w mannitol.

32-34. (canceled)

35. The process according to claim 20, wherein the sterile solution contains about 5% w/w pyridoxal 5′-phosphate, about 1.5% w/w of the sodium hydroxide and about 2.8% w/w of the mannitol.

36. The process according to claim 20, wherein the sterile solution contains about 250 mg pyridoxal 5′-phosphate, about 80.5 mg sodium hydroxide and about 150 mg of mannitol.

37. A kit useful for preparing an injectable formulation, comprising
(a) instructions for preparing the injectable formulation:
(b) a container comprising the lyophilized formulation of pyridoxal 5′-phosphate of according to claim 1; and
(c) a container comprising a sterile carrier suitable for intravenous administration.

38. The kit according to claim 37, wherein the sterile carrier is water for injection.

39. The kit according to claim 37, further comprising a container for said injectable formulation, said container sized to facilitate preparation of a selected volume and concentration of said formulation.

40. (canceled)

41. A method of treating a patient in need of treatment with pyridoxal-5-phosphate comprising intravenously administering the injectable formulation according to claim 18.

42. The method according to claim 41, wherein said patient is in need of reduction of the incidence of nausea and vomiting associated with the oral administration of pyridoxal 5′-phosphate or a pharmaceutically acceptable salt thereof.

43. (canceled)

44. The method according to claim 41, wherein said patient undergoing a surgical procedure.

45. The method according to claim 44, wherein the surgical procedure is coronary artery bypass grafting (CABG) or percutaneous coronary intervention (PCI).

46-47. (canceled)
